(19) United States
(12) Patent Application Publication
(10) Pub. No.: US 2026/0306457 A1
(43) Pub. Date: Oct. 8, 2026

(54) SULFURIZED IMIDAZOLES IN OPHTHALMIC FORMULATIONS

(71) Applicant: GENEIII BIOTECH COMPANY LTD., Nanjing (CN)

(72) Inventors: Cai Rong Wang, Hacienda Heights, CA (US); Wei Ding, Reno, NV (US)

(21) Appl. No.: 19/381,305

(22) Filed: Nov. 6, 2025

Related U.S. Application Data
(63) Continuation-in-part of application No. 19/172,995, filed on Apr. 8, 2025.

(51) Int. Cl. A61K 31/4172; A61K 9/00; A61K 9/08; A61K 31/132; A61K 31/14; A61K 31/195; A61K 31/205; A61K 31/221; A61K 31/282; A61K 31/40; A61K 31/452; A61K 31/7068; A61K 31/7072; A61K 47/02; A61K 47/10; A61K 47/18; A61K 47/22; A61K 47/36; A61P 27/04

(52) U.S. Cl. CPC A61K 31/4172; A61K 9/0048; A61K 9/08; A61K 31/132; A61K 31/14; A61K 31/195; A61K 31/205; A61K 31/221; A61K 31/282; A61K 31/40; A61K 31/452; A61K 31/7068; A61K 31/7072; A61K 47/02; A61K 47/10; A61K 47/18; A61K 47/183; A61K 47/22; A61K 47/36; A61P 27/04

(57) ABSTRACT

In the embodiments, ophthalmic formulations comprise 0.001-2% (w/v) of L-Ergothioneine or derivatives of L-Ergothioneine, optionally combined with compatible toxicity agents. L-ergothioneine or derivatives of L-Ergothioneine in the ophthalmic formulation is: (1) an active pharmaceutical ingredient (API) for treating oxidative stress-related ocular disorders; and (2) an inactive excipient for use as a stabilizing excipient to enhance bioavailability and prolong shelf-life of co-administered APIs. L-ergothioneine or derivatives of L-ergothioneine in the ophthalmic solution, when applied on an ocular surface, exhibit: improved corneal permeability; reduced degradation of thermolabile components; and synergistic therapeutic effects when used with other neuroprotective agents, as the co-formulated APIs, in the ophthalmic formulations.

### CROSS-REFERENCE TO RELATED APPLICATIONS

This application is a continuation-in-part of U.S. patent application Ser. No. 19/172,995, filed Apr. 8, 2025, the contents of which are herein incorporated by reference.

### BACKGROUND OF THE INVENTION

### 1. Field of the Invention

The compositions and methods herein are directed to pharmaceutical formulations for treating ocular diseases or alleviating symptoms associated with ocular diseases.

### 2. Description of Prior Art and Related Information

Over the past several decades, more than a dozen organic compounds have been developed and used as active ingredients of eye drops. Many active ingredients and preservatives have upper concentration level limits based on the considerations of toxicities for certain formulations. The search for active pharmaceutical ingredients and inactive excipients of eye drops with higher potency and lower toxicity is a focus of attention. Currently, most eye drops on the market are formulated with specific active ingredients for specific effects and can be divided into four categories: treatment of redness in eye, lubricating, tears in the eye, and allergies in the eyes.

The eye, particularly the ocular surface, is a highly vulnerable tissue, constantly exposed to environmental stressors such as ultraviolet radiation, dust, pollutants, and mechanical friction. This exposure generates a large amount of reactive oxygen species (ROS), leading to oxidative stress, inflammation, and cell damage, which in turn can cause diseases such as dry eye and corneal degeneration. Therefore, the eye has a vital need for a strong antioxidant defense system.

L-Ergothioneine (EGT), due to its unique stability and high antioxidant capacity, plays an important role in maintaining the health of ocular surface. It is particularly abundant in the lens and cornea of the eye, providing a strong theoretical basis for the exploration of our study

What is needed are the compositions and methods described herein, namely, an ophthalmic formulation containing L-Ergothioneine or derivatives of L-Ergothioneine (e.g., the application of L-Ergothioneine in eye drops), that are loaded at a concentration lower than the upper concentration level limits, thereby avoiding potentially toxic levels, while exhibiting higher potency in: (1) increasing lubrication; and (2) reducing redness, tears, and allergies.

### SUMMARY OF THE INVENTION

In a variant, an ophthalmic formulation comprises: a pharmaceutically acceptable aqueous delivery vehicle; and L-Ergothioneine (EGT) in a concentration of 0.005% to 2.000% (w/v) in the pharmaceutically acceptable aqueous delivery vehicle, wherein the EGT is: an active ingredient for treating an oxidative stress-related ocular disorder or disease; or an inactive excipient to inhibit degradation of a co-formulated active ingredient.

In a variant, the co-formulated active ingredient is selected from the group consisting of: nucleotides, acetylcholine, tetraethylammonium chloride, spermine, L-carnitine, sulpiride, homostachydrine, cytarabine, gemcitabine, gabapentin, oxaliplatin, metformin, cyclosporine, and lifitegrast.

In a variant, EGT scavenges hydroxyl and peroxynitrite via a non-enzymatic redox cycle, such that: the hydroxyl is reduced to water and EGT is oxidized to a sulfate; and the peroxynitrite is reduced to nitrate and EGT is oxidized to a sulfate.

In a variant, EGT coordinates to a metal selected from iron, copper, and other transition metals in an ocular surface, thereby inhibiting catalysis of oxidative degradation of labile drugs by: forming a complex between EGT and the metal, wherein the metal is in a reduced state; and reducing an oxidation potential of the metal due to the formation of the complex, thereby suppressing formation of metal-oxygen adducts for deriving reactive oxygen species; wherein the complex comprises: (i) two units of EGT binding to 1 unit of the metal or (ii) 1 unit of EGT and 1 unit of an initial ligand core binding to 1 unit of the metal.

In a variant, EGT as the inactive excipient in the formulation is reducing reactive oxygen species (ROS) in corneal epithelial cells.

In a variant, EGT is 0.5% to 1.0% (w/v) of the formulation, when functioning as an active ingredient; EGT is 0.005% to 0.5% (w/v) of the formulation, when functioning as an inactive excipient.

In a variant, the oxidative stress-related ocular disorder or disease comprises: allergies, irritated eyes, red eye, age-related macular degeneration; cataracts; glaucoma; diabetic retinopathy; dry eye syndrome; and retinitis pigmentosa.

In a variant, the co-formulated active ingredient is transported in the ocular tissue by OCTN1, thereby increasing bioavailability of the co-formulated active ingredient in the ocular tissue, wherein OCTN1 is inductively expressed by presence of EGT introduced into the ocular tissue.

In a variant, the formulation further comprises one or more of: glutathione, Ubiquinone, lutein; and ethylenediaminetetraacetic acid.

In a variant, EGT has a chemical formula of C₉H₁₅N₃O₂S, where the carbon-sulfur bond is in thione form (C═S) over thiolate form (C—S⁻), in the pharmaceutically acceptable aqueous delivery vehicle.

In a variant, the formulation comprises: 0.5% (w/v) of EGT as the active ingredient; 0.05% (w/v); 0.01% (w/v), and 0% (w/v) of sodium hyaluronate; trace amounts of taurine; sodium chloride for adjusting the osmotic pressure of eye drops; and the pharmaceutically acceptable aqueous delivery vehicle.

In a variant, an ophthalmic formulation comprises: a pharmaceutically acceptable aqueous delivery vehicle; and a derivative of L-Ergothioneine (EGT) in a concentration of 0.005% to 3.000% (w/v) in the pharmaceutically acceptable aqueous delivery vehicle, wherein the derivative of EGT is: an active ingredient for treating an oxidative stress-related ocular disorder or disease; or an inactive excipient to inhibit degradation of a co-formulated active ingredient, wherein the derivative of EGT is:

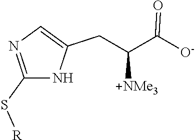

where R=CH₃, CH₂CH₃, CH₂CH₂CH₃, CH₃CHCH₃, CH₂—X, and X=1-indole, 4-Indole, 7-indole, 1-imidazole, 2-imidazole, 3-imidazole, 4-imidazole, 5-imidazole, benzene, 2-oxazole, 3-oxazole, 4-oxazole, 2-thiazole, 3-thiazole, 4-thiazole, 2-pyridine, 6-pyridine, 1-pyrrole, 2-pyrrole, and 3-pyrrole.

In a variant, wherein the co-formulated active ingredient is selected from the group consisting of: nucleotides, acetylcholine, tetraethylammonium chloride, spermine, L-carnitine, sulpiride, homostachydrine, cytarabine, gemcitabine, gabapentin, oxaliplatin, metformin, cyclosporine, and lifitegrast.

In a variant, the derivative of EGT scavenges hydroxyl, such that the hydroxyl is reduced to water and the derivative of EGT is oxidized to a sulfoxide.

In a variant, the derivative of EGT as the inactive excipient in the formulation is decreasing reactive oxygen species (ROS) in corneal epithelial cells.

In a variant, the derivative of EGT is 1.0% to 3.0% (w/v) of the formulation, when functioning as an active ingredient; and the derivative of EGT is 0.005% to 1.0% (w/v), when functioning as an inactive excipient.

In a variant, the oxidative stress-related ocular disorder or disease comprises: allergies, irritated eyes, red eye, age-related macular degeneration; cataracts; glaucoma; diabetic retinopathy; dry eye syndrome; and retinitis pigmentosa.

In a variant, a method of treating an oxidative stress-related ocular condition, disorder, or disease in a subject, wherein the method comprises: administering a formulation to said subject, wherein the formulation comprises L-Ergothioneine (EGT) or derivative of EGT in a concentration of 0.005% to 2.000% (w/v) in a pharmaceutically acceptable aqueous delivery vehicle, wherein: EGT or the derivative of EGT is: an active ingredient for treating an oxidative stress-related ocular disorder or disease; or an inactive excipient to inhibit degradation of a co-formulated active ingredient.to the ocular surface of the subject.

In a variant, EGT or the derivative of EGT scavenges reactive oxygen species via a non-enzymatic redox cycle.

In a variant, wherein EGT or the derivative of EGT coordinates to a metal selected from iron, copper, and other transition metals in an ocular surface, thereby inhibiting catalysis of oxidative degradation of labile drugs and decreasing reactive oxygen species (ROS) in corneal epithelial cells.

### BRIEF DESCRIPTION OF THE DRAWINGS

FIG. 1 is a depiction of canonical structures of L-Ergothioneine (EGT).

FIG. 2 is a depiction of EGT and derivatives of EGT and the scavenging of reactive oxygen species.

FIG. 3 is a depiction of EGT binding to free metal ions.

FIG. 4 is a depiction of EGT binding to iron from lipoxygenase.

FIG. 5 is a depiction of EGT binding to iron from Cytochrome P450.

FIG. 6 is a depiction of a questionnaire plot.

FIG. 7 is a depiction of a OSDI Rating Scale/Score plot.

FIG. 8 is a depiction of a visual fatigue scale/score plot.

FIG. 9 is a depiction of the first tear film beak-up time plot for the left eye.

FIG. 10 is a depiction of the first tear film beak-up time plot for the right eye.

### DETAILED DESCRIPTION OF THE PREFERRED EMBODIMENTS AND BEST MODE OF INVENTION

In the compositions and methods herein, the ophthalmic formulation herein can be introduced into a physiological environment, as eye drops added to ocular tissue of a human subject. The ophthalmic formulation herein comprises: (1) a pharmaceutically acceptable aqueous delivery vehicle (e.g., water as a solvent for dissolving tonic agents, buffers, preservatives, viscosity enhancers, stabilizers, pH adjusters, penetration enhancers, local anesthetics, restorative agents, surfactants, and thickeners); (2) L-Ergothioneine (EGT) or derivatives of EGT dissolved in the water phase of the pharmaceutically acceptable aqueous delivery vehicle; (3) one or more co-formulated active ingredients (nucleotides (e.g., uridine), acetylcholine, tetraethylammonium chloride, spermine, L-carnitine, sulpiride, homostachydrine, cytarabine, gemcitabine, gabapentin, oxaliplatin, metformin, cyclosporine, and Lifitegrast); and (4) other ingredients selected from one or more of: (a) sodium chloride or other sodium salts for controlling osmotic pressure; (b) stabilizers (ethylenediaminetetraacetic acid); (c) hyaluronates; (d) taurine (2-aminoethanesulfonic acid); and (e) co-antioxidants (glutathione, ubiquinone, and lutein).

In the compositions and methods herein, EGT or derivatives of EGT are introduced to ocular tissue via an ophthalmic formulation, such as eye drops. EGT or derivatives of EGT in the ophthalmic formulation can behave as: (1) an active pharmaceutical ingredient (API) for treating or alleviating symptoms associated with oxidative stress-related ocular disorders (e.g., allergies, irritated eyes, red eye, age-related macular degeneration, cataracts, glaucoma, diabetic retinopathy, dry eye syndrome; and retinitis pigmentosa, and UV-induced corneal damage); and (2) an inactive excipient for use, as a stabilizing excipient, to: (a) enhance the bioavailability of co-administered active pharmaceutical ingredients; (b) prolong the shelf-life of co-administered active pharmaceutical ingredients; (c) increase the stability of the ophthalmic formulation herein; and (d) increase compatibility of EGT or EGT derivatives with other ocular therapeutics. The combination of API and excipient behavior of EGT or derivatives of EGT can: improve the potency of eye drops and reduce the toxicity of eye drops, in comparison to eye drops absent of EGT or derivatives of EGT. This can be attributed to: reduction of irritating excipients; the low-toxicity, antioxidant, and anti-inflammatory properties of EGT or derivative of EGT; and the clinical safety, which is described below.

In the compositions and methods herein, the ophthalmic formulations herein, which contain EGT or derivatives of EGT in the pharmaceutically acceptable aqueous delivery vehicle have a viscosity, surface activity, and surface tension adjustments that aid in the targeted delivery of EGT or derivatives of EGT to reduce oxidative stress in corneal epithelial cells. The amount of EGT or derivatives of EGT in the ophthalmic formulation herein can determine whether EGT or a derivative of EGT is an API or excipient. For example, the concentration of EGT in the ophthalmic formulation herein is: (1) 0.5% to 1.0% (w/v), when functioning as an active pharmaceutical ingredient; or (2) 0.005% to 0.5% (w/v) when functioning as an inactive excipient. For example, the ophthalmic formulations herein comprise: 98.0-99.5% (w/v) of water; 0.1-0.5% (w/v) of triethylamine; 0.05-2.0% (w/v) of L-carnitine; 0.01-0.1% (w/v) of Spermine, and 0.1-1.0% (w/v) of acetylcholine, in combination with: (1) 0.5% to 1.0% (w/v); or (2) 0.005% to 0.5% (w/v) of EGT. EGT is dissolved in the pharmaceutically acceptable aqueous delivery vehicle, which is predominantly water and contributes at least 85 to 90% (w/v) water to the formulation. The water content of the pharmaceutically acceptable aqueous delivery vehicle dissolves one or more of a tonic agent, buffer, preservative, viscosity enhancer, stabilizer, pH adjuster, penetration enhancer, local anesthetic, restorative agent, surfactant, and thickeners, to (1) make the pharmaceutically acceptable aqueous delivery vehicle, to which (2) EGT is added. To achieve the same level of alleviation of ocular symptoms, lower amounts of EGT or derivatives of EGT are used in the ophthalmic formulations herein, in comparison to other API eye treatments, especially with the consideration of the same toxicity level.

In the compositions and methods herein, EGT or derivatives of EGT can perform the functions of: (1) scavenging reactive oxygen species (ROS), such as free radicals (e.g., hydroxyl radical) and free radical surrogates (e.g., peroxynitrite (ONOO⁻) or peroxynitrous acid (ONOOH)), more effectively than butylated hydroxytoluene (BHT) and 1,2,3-benzotriazole (BHA), with minimal cytotoxicity; (2) exhibiting strong antioxidant activity to consume ROS that can oxidatively degrade co-formulated active ingredients; (3) inhibiting inflammation, thereby making the ophthalmic formulation herein the particularly suitable for coping with corneal oxidative stress caused by light loss and dry eye syndrome; (4) exhibiting dual-phase stabilization to inhibit degradation of labile active ingredients (e.g., pharmaceutical ingredients prone to chemical degradation (e.g., oxidation, hydrolysis, and photolysis) or changes in physical properties (e.g., aggregation and crystallization)) in aqueous solutions, without additional preservatives, such that two immiscible phases maintain interface stability between a non-polar phase (oil) and polar phase (water) with emulsifiers and stabilizer, as exhibited in micelles, emulsions, and/or interactions between: (a) an inner mucus layer and middle aqueous layer and (b) an outer lipid layer and middle aqueous layer, in tear films; and (5) enhancing bioavailability of the co-formulated activate ingredients, via the affinity of EGT for OCTN1 transporters in corneal epithelial cells (i.e., EGT induced expression of OCTN1 transporters), thereby facilitating targeted delivery of co-formulated active ingredients (i.e., transporting the co-formulated active ingredient in ocular tissue).

In the composition and methods herein, the preferred embodiment of the ophthalmic formulations herein comprises EGT. More particularly, 0.001-2.000% (w/v) of L-Ergothioneine (EGT) can be optionally combined with compatible toxicity agents. Compatible cytotoxicity agents are a combination of ingredients that coexist in a preparation, such as tablet, eye drop, or other formulation, associated with a certain degree of toxicity. The combination of ingredients require specific formulation design to ensure chemical and physical compatibility, while managing toxicity. For example, 0.005-0.01% (w/v) of BAK and 0.01-0.05% (w/v) of EDTA can reduce metal ion oxidizing and increase antibacterial effects.

In the compositions and methods herein, other embodiments of the ophthalmic formulations herein comprise derivatives of EGT. More particularly, 0.002-3.000% (w/v) of derivatives of EGT can be optionally combined with compatible toxicity agents. A higher amount of derivatives of EGT, in comparison to EGT, may be necessary to achieve the same therapeutic effect of EGT. This may be attributed to an active group of the EGT derivatives being obscured and not compensated by other mechanisms, such as targeting; and/or efficacy enhancement by optimizing pharmacokinetics.

In the compositions and methods herein, EGT or derivatives of EGT in the ophthalmic solution, when applied on an ocular surface, lead to: improved corneal permeability; reduced degradation of thermolabile components; and synergistic therapeutic effects when used with other neuroprotective agents in the ophthalmic formulations. The thermolabile components (proteins and peptide drugs, certain antibiotics, probiotics, nucleic acid drugs) are prone to degradation, inactivation, or physical property changes, at high temperature environments (e.g., addition of heat). Neuroprotective agents protect neural cells from damage induced by glutamine or oxidative stress.

In the compositions and methods herein, the ophthalmic formulations herein can alternatively comprise EGT, in combination with other antioxidants (e.g., glutathione or lutein) for synergistic effects, in treating oxidative-stress related conditions in ocular tissue.

In the compositions and methods herein, the ophthalmic formulations herein can alternatively comprise EGT, in combination with stabilizers (e.g., ethylenediaminetetraacetic acid (EDTA)), as a preventative replacement in multi-dose eye drops.

In the compositions and methods herein, the ophthalmic formulations herein can alternatively be reformulated as nasal sprays or intraocular injections.

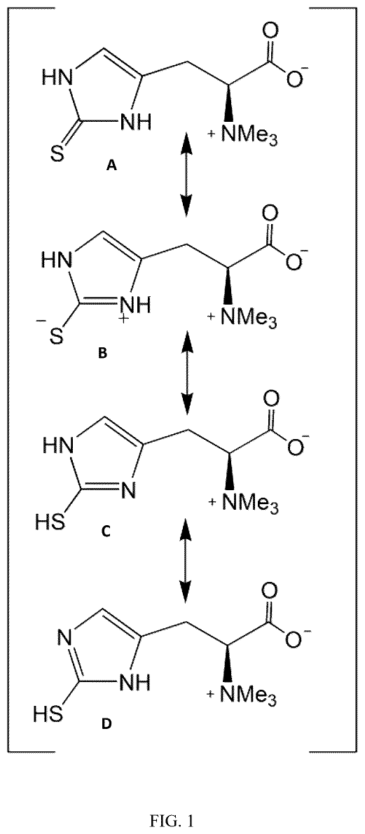

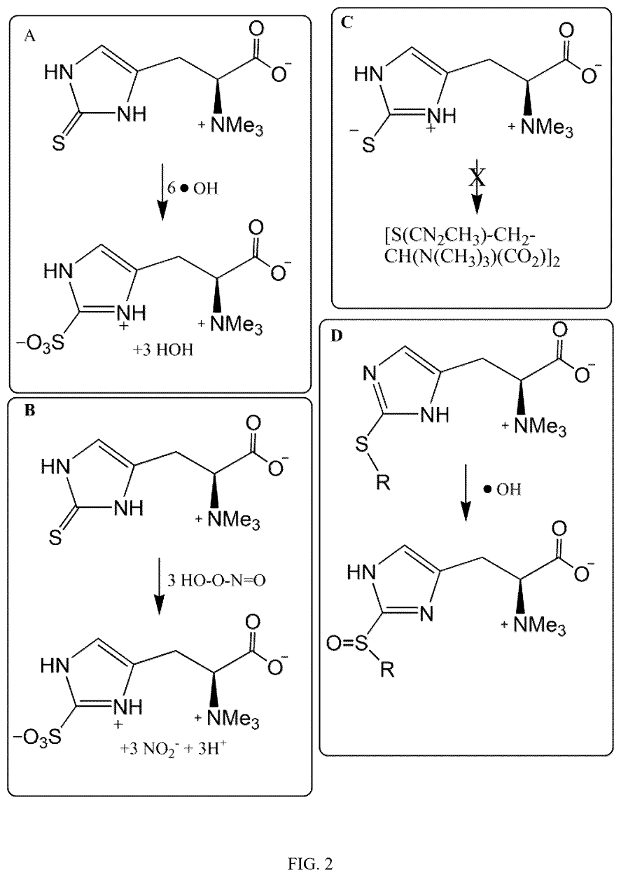

In the compositions and methods herein, the ophthalmic formulations herein comprising EGT are a preferred embodiment, wherein EGT has a thiourea group, which indicates that a thione (C═S, which is A in FIG. 1) canonical structure is favored over thiol (C—SH, which is C and D in FIG. 1) or thiolate (C—S⁻, which is B in FIG. 1) canonical structures, thereby allowing for the EGT to be an active pharmaceutical ingredient and an inactive excipient. Without being bound to a theory, thiourea of EGT, which has C═S, can scavenge of hydroxyl radicals, peroxynitrite, and peroxynitrous acid, via a non-enzymatic redox cycle. Enzymes, such as lipoxygenase and cytochrome P450, can form metal-oxygen adducts, to derive reactive oxygen species (e.g., hydroxyl radicals, peroxynitrite, and peroxynitrous acid) that react with substrates, which lead to oxidative degradations of said substrates that is associated with oxidative stress via a redox cycle. The scavenging of, for example, hydroxyl radicals and peroxynitrous acid, via non-enzymatic redox cycle, as made operable by EGT, consumes hydroxyl radicals and peroxynitrous acid to: (1) convert the thione (C═S) to sulfate (C—SO₃⁻) and obtain water (see A in FIG. 2); and (2) convert the thione (C═S) to sulfate (C—SO₃⁻) and obtain nitrate (NO₂⁻), and protons (see B in FIG. 2), respectively. If the canonical structure of EGT is not thione, but instead a thiolate, the observed consumption of ROS would not occur. Thiolate, such as C in FIG. 2, is an anion that has a high oxidation potential to form a thiyl radical that favors formation of disulfide. Said thiyl radical is not as readily attainable in thione. Disulfide is not observed, when the ophthalmic formulation herein is added to an ocular tissue, which suggests that the thione of a thiourea (C═S) is scavenging the ROS.

In the compositions and methods herein, the ophthalmic formulation comprises a derivative of EGT. Derivatives of EGT have the formula of RS(C(N)(NH)(CHC))—CH₂—CH(N(CH₃)₃)(CO₂), where R=CH₃, CH₂CH₃, CH₂CH₂CH₃, CH₃CHCH₃, CH₂—X, and X=1-indole, 4-Indole, 7-indole, 1-imidazole, 2-imidazole, 3-imidazole, 4-imidazole, 5-imidazole, benzene, 2-oxazole, 3-oxazole, 4-oxazole, 2-thiazole, 3-thiazole, 4-thiazole, 2-pyridine, 6-pyridine, 1-pyrrole, 2-pyrrole, and 3-pyrrole. EGT has the formula of S(C(NH)(NH)(CHC))—CH₂—CH(N(CH₃)₃)(CO₂). Derivatives of EGT are thioethers, which have a more similar valence to thione than thiolate, that scavenge ROS to yield sulfoxide (RS(O)—(C(N)(NH)(CHC))—CH₂—CH(N(CH₃)₃)(CO₂), see D of FIG. 2). Without being bound to a theory, the valence of the sulfur attached to the imidazole ring can impact the oxidation of the sulfur, which leads to the scavenging of ROS.

In the compositions and methods herein, the thione (C═S) of the EGT of the ophthalmic formulation can suppress the formation of ROS that lead to the oxidative degradation of labile drugs, such as co-formulated active ingredients, in the ophthalmic formulation. Without being bound to a theory, the sulfur of the thione is a donating atom that can form chelation complexes with a metal center. This chelation complex lowers the oxidation potential of the chelated metal center, thereby suppressing redox processes that lead to the formation of metal oxygen adducts for deriving ROS.

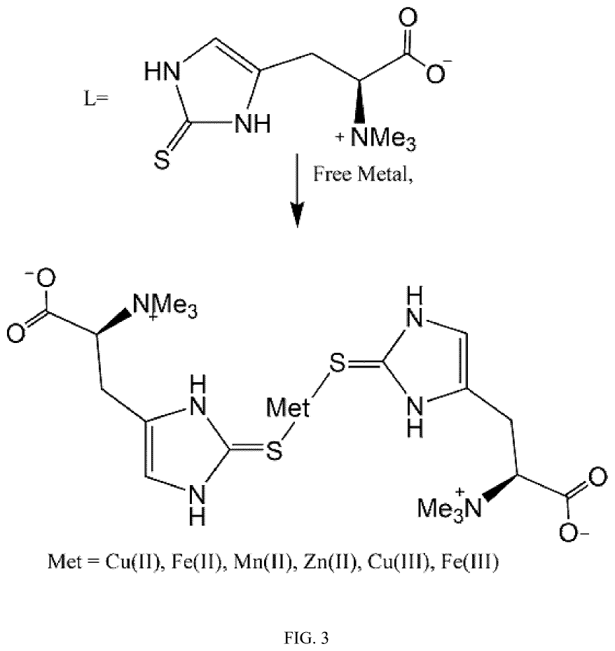

In the compositions and methods herein, two units of EGT can bind to free ions, such as Cu(II), Fe(II), Mn(II), Zn(II), Cu(III), and Fe(III), wherein the sulfur of the thione (C═S) is binding to said free ions, as depicted in FIG. 3. The chelation complex is FIG. 3 does not have sufficient oxidation potential to form metal-oxygen adducts for deriving ROS.

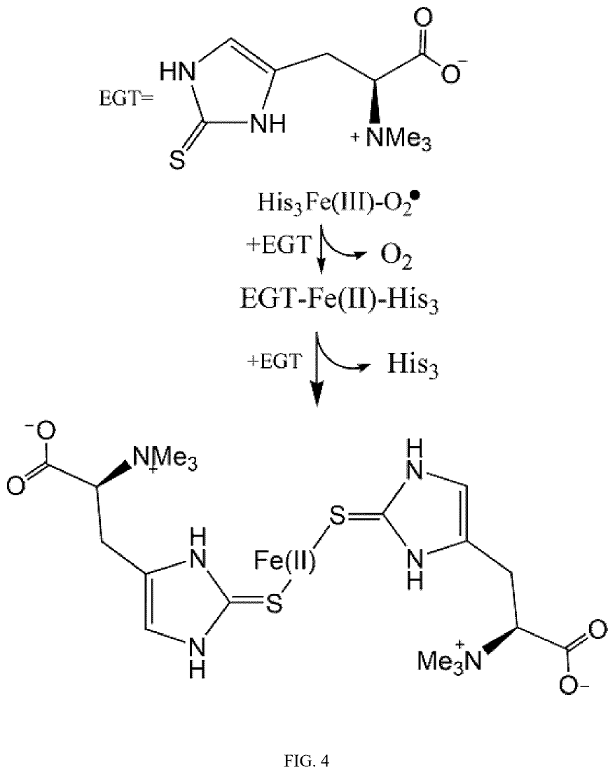

In the compositions and methods herein, two units of EGT can bind to Fe(II) from lipoxygenase-superoxide (His₃-Fe(III)-O₂●), thereby suppressing the persistence of a metal-oxygen adduct for deriving or behaving as a ROS. More particularly, the sulfur of the thione (C═S) from the first unit of EGT induces release of dioxygen, with concurrent reduction from Fe(III) to Fe(II), when the first unit of EGT binds to the iron center, to yield His₃-Fe(II)-EGT. The second unit of EGT binds to the Fe(II) site, thereby de-ligating the histidine core from the iron in lipoxygenase, to yield EGT-Fe(II)-EGT, as depicted in FIG. 4. The histidine core of lipoxygenase is a strong nitrogen-atom donating ligand for binding to Fe(II) and imparting increased oxidation potential that readily forms metal-oxygen adducts that lead to ROS. The chelation complex containing two units of EGT bound to Fe(II), as depicted in FIG. 4, does not form metal oxygen adducts. This suggests that: (1) EGT can behave as a ligand that binds to Fe(II) over the histidine core of lipoxygenase; and (2) and the two units of EGT lowers the oxidation potential of Fe(II), to suppress redox processes that form metal oxygen adducts for deriving ROS.

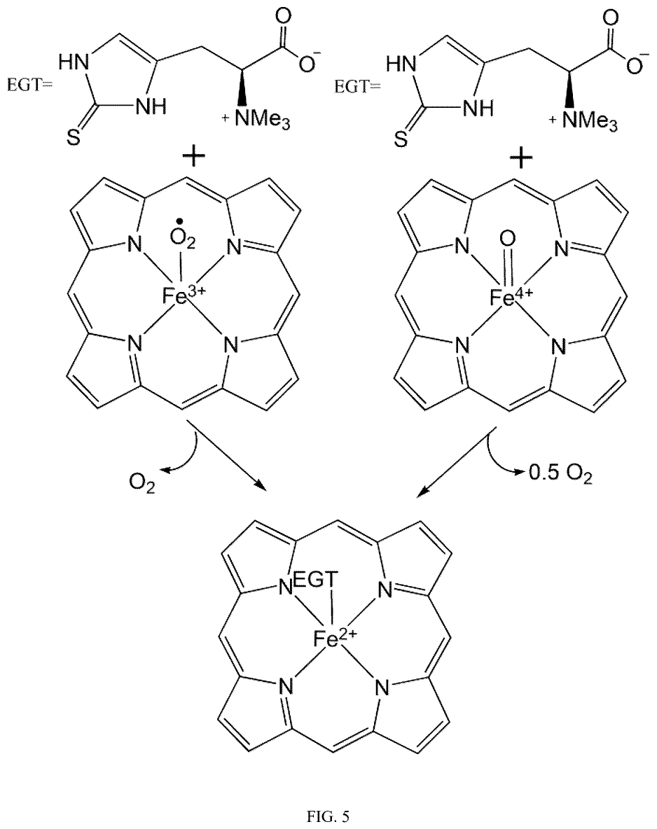

In the composition and methods herein, a unit of EGT can bind to Fe(II) from Cytochrome P450, thereby suppressing the persistence of a metal-oxygen adduct that can behave as or derive ROS. Cytochrome P450 has a porphyrin core around Fe, as depicted in FIG. 5, wherein Fe can bind to a single unit of EGT. When a metal oxygen adduct is formed in cytochrome P450, the Fe(III)-superoxide or Fe(IV)=O can form, which are ROS or derive ROS that oxidatively degrade labile drugs or lead to oxidative stress in the ocular tissue. While the porphyrin core is not readily de-ligated, the sulfur atom of the thione of EGT can bind to an oxidized iron site (Fe(III) or Fe(IV)) to release dioxygen, and concurrently reduce the iron site to Fe(II), thereby forming a chelation complex of EGT-Fe(II). The chelation complex of EGT-Fe(II) lowers the oxidation potential of Fe(II), to suppress redox processes that form metal oxygen adducts for deriving ROS.

In the compositions and methods herein, the ophthalmic formulation comprises EGT and co-formulated active ingredients, wherein EGT induces expression of transporter OCTN1. Transporter OCTN1 can transport co-formulated active ingredients to ocular tissue, when the ophthalmic formulation comprising EGT and co-formulated active ingredients is introduced into ocular tissue. This transport of co-formulated active ingredients to ocular tissue is associated with the increase of the bioavailability of the co-formulated active ingredients in the ocular tissue. Additionally, EGT can induce upregulation of Nuclear Factor Erythroid 2-Related Factor 2 (Nrf2), thereby expressing antioxidant proteins that protect against ROS.

In the compositions and methods herein, the ophthalmic formulation comprises EGT and the pharmaceutically acceptable aqueous delivery vehicle, wherein the pharmaceutically acceptable aqueous delivery vehicle is prepared by step-by-step dissolutions of excipients selected from the group consisting of a tonic agent, buffer, preservative, viscosity enhancer, stabilizer, pH adjuster, penetration enhancer, local anesthetic, restorative agent, surfactant, and thickeners. Sterilization and strict processing of the pharmaceutically acceptable aqueous delivery vehicle ensure the functionality and sterility of the excipients in the pharmaceutically acceptable aqueous delivery vehicle, to which EGT or derivatives of EGT is added. This process balances the stability of active ingredients, preparation safety and production efficiency, and complies with the requirements of Good Manufacturing Practice (GMP) (current Good Manufacturing Practice cGMP) for pharmaceutical production.

In the compositions and methods herein, the preferred embodiment of the ophthalmic formulations herein contains L-Ergothioneine (EGT), as represented by Formula 1. L-Ergothioneine is a derivatized histidine, wherein the imidazole is disubstituted at the 2-position and the 4-position and the amine is trimethylated. More specifically, the imidazole (C(NH)(NH)(CHC)) is sulfurized(S) at the 2-position and connected to methylene (CH₂), wherein methylene is connected to a methine group (CH), wherein methine is connected to trimethylammonium (N(CH₃) 3) and carboxylate (CO₂). The trimethylammonium group, which is cationic by virtue of being a quaternary amine (without any protons attached to the nitrogen); and the carboxylate, which is anionic by virtue of being in a deprotonated state, lead a betaine construct for promoting surfactant and emulsification properties. Surfactant and emulsification properties aid in: (1) dissolving L-Ergothioneine in water of the ophthalmic formulation; and (2) releasing L-Ergothioneine from the ophthalmic formulation, when introduced into ocular tissue. At the 2-position of imidazole, sulfur can be in thione form (C═S) or thiolate (C—S—) or thiol (C—SH) forms. However, thione is the favored canonical structure in the ophthalmic formulation and in the ocular tissue, when the formulation containing L-Ergothioneine is introduced into the ocular tissue. In thione form, L-Ergothioneine is able to scavenge reactive species and bind to metal centers (e.g., free ion or accessible faces of a transition metal complex). The antioxidant properties of thione-imidazole (i.e., chemo-selective oxidation of the sulfur of the thione-imidazole) and binding behavior of L-Ergothioneine in thione form to metal centers can allow for a controlled mechanism to be operable in the ocular tissue, when introduced therein from the ophthalmic formulation.

S(C(NH)(NH)(CHC))—CH₂—CH(N(CH₃)₃)(CO₂) [Formula 1]

In the compositions and methods herein, other embodiments of the ophthalmic formulation includes derivatives of L-Ergothioneine, as represented by Formula 2. Derivatives of L-Ergothioneine comprise imidazole that is disubstituted at the 2-position and the 4-position and the amine is trimethylated. More specifically, the imidazole (C(N)(NH)(CHC)) is connected to SR at the 2-position and connected to methylene (CH₂), wherein methylene is connected to a methine group (CH), wherein methine is connected to trimethylammonium (N(CH₃)₃) and carboxylate (CO₂). The trimethylammonium group, which is cationic by virtue of being a quaternary amine (without any protons attached to the nitrogen); and the carboxylate, which is anionic by virtue of being in a deprotonated state, lead a betaine construct for promoting surfactant and emulsification properties. Surfactant and emulsification properties aid in: (1) dissolving the derivatives of L-Ergothioneine in water of the ophthalmic formulation; and (2) releasing the derivatives of L-Ergothioneine from the ophthalmic formulation, when introduced into ocular tissue.

RS(C(N)(NH)(CHC))—CH₂—CH(N(CH₃)₃)(CO₂) [Formula 2]

In the compositions and methods herein, at the 2-position of imidazole, the R group of SR of the derivatives of L-Ergothioneine are functional groups that make thioethers, where: R=CH₃, CH₂CH₃, CH₂CH₂CH₃, CH₃CHCH₃, CH₂—X, and X=1-indole, 4-Indole, 7-indole, 1-imidazole, 2-imidazole, 3-imidazole, 4-imidazole, 5-imidazole, benzene, 2-oxazole, 3-oxazole, 4-oxazole, 2-thiazole, 3-thiazole, 4-thiazole, 2-pyridine, 6-pyridine, 1-pyrrole, 2-pyrrole, and 3-pyrrole. While the connectivity of sulfur to the imidazole in Formula 2 does not make a thione connectivity as a feasible canonical structure, the thioethers of Formula 2 do not yield a thiolate or thiyl. In thioether form, L-Ergothioneine is able to scavenge reactive species and bind to metal centers (e.g., free ion or accessible faces of a transition metal complex). The antioxidant properties of thioether-imidazole (i.e., chemo-selective oxidation of the sulfur of the thioether-imidazole) and binding behavior of the derivatives of L-Ergothioneine in thioether form to metal centers can allow for a controlled mechanism to be operable in the ocular tissue, when introduced therein from the ophthalmic formulation.

In the compositions and methods herein, at the 2-position of imidazole, the R group of SR of the derivatives of L-Ergothioneine is not H. Thus, sulfhydryl (SH) is not attached to the 2-position of imidazole. Sulfhydryl can donate a proton (H⁺) to yield a thiolate anion (⁻S—(C(N)(NH)(CHC))—) or hydrogen radical (H●) to yield thiyl radical (●S—(C(N)(NH)(CHC))—). Thiolate and thiyl are canonical structures that are far more reactive than the thione, and therefore not as easy to control the reactivity, in comparison to the thione form. This can lead to redox processes that can reduce or entirely suppress the operability of the controlled mechanism, as indicated above.

Transport Mechanism of EGT from Ocular Surface to Retina Via OCTN1

Ergothioneine (EGT) has excellent antioxidant and anti-inflammatory properties and effects, with more effective in reducing mitochondrial oxidative stress and other possible damage. While research on its individual effects is extensive, there's limited direct research on the transport kinetics of EGT from ocular surface to inside of eyeball. However, as described herein, it is highly plausible that EGT could work together with other ingredients such as spermine and prostaglandin analogs to provide enhanced and more comprehensive cellular protection in eyes.

Ergothioneine (EGT) is a unique, sulfur-containing amino acid derivative primarily found in mushrooms and some other food sources. It is recognized for its high chemical stability and powerful antioxidant properties. Its specific transporter protein, OCTN1 (in some literatures, it is called ETT meaning EGT (in some literatures just simplified as ET) specific transporter), allows it to accumulate in tissues that are prone to oxidative damage, like the liver, kidneys, brain, and eyes.

Ergothioneine transports into cells, including certain cell lines like HeLa cells, is mediated primarily by the high specific transporter OCTN1 (Organic Cation Transporter Novel Type 1), also known as SLC22A4. However, there are only several direct, published kinetic data available for ergothioneine transport via OCTN1 specifically in HCLE/HCjE (Immortalized Human Corneal-Limbal/Corneal-Conjunctival) cells. The available research focuses on other cell types, like HEK293 cells, which are often used to express and study transporter kinetics.

Aspects of the present invention confirm that EGT is essential for ocular health and that its dedicated transporter, OCTN1, is present in these key ocular surface cell lines. One finding is that OCTN1/2 are primarily localized to the apical membrane of these cells (epithelium), which has important implications for future product development, especially for the development of glaucoma eye drops, as discussed in greater detail below. While research results confirm the existence of this transport system, the specific kinetic parameters (Km and Vmax) for human HCLE/HCjE cells are still unknown. Therefore, aspects of the present invention provide a strategic roadmap for first validating the transport kinetics, then demonstrating the functional efficacy of EGT, and ultimately translating these findings into clinically and commercially viable strategies.

EGT, due to its unique stability and high antioxidant capacity, plays an important role in maintaining the health of ocular surface. It is particularly abundant in the lens and cornea of the eye, providing a strong theoretical basis for exploration and study.

EGT's uniqueness lies not only in its chemical properties but also in the specialized transport system that the human body has evolved it. OCTN1 (organic transporter 1, also known as SLC22A4) has been identified as the primary transporter of EGT. Although OCTN1 has multi-specificity and can transport other molecules such as carnitine, studies have shown that EGT is its preferred endogenous substrate. Therefore, OCTN1 has also been functionally designated the EGT transporter (ETT). Unlike other antioxidants that diffuse passively, EGT uptake is an active, regulated process, indicating that cells have a high physiological demand for EGT.

Human limbal epithelial cells (HCLE) and human conjunctival epithelial cells (HCjE) were selected as research models for their high biological relevance. HCLE cells contain limbal stem cells (LESCs), which are responsible for the continuous renewal and repair of the corneal epithelium, maintaining corneal integrity. HCjE cells, a crucial component of conjunctival tissue, function through mucin-secreting goblet cells to lubricate the eye, maintain tear film stability, and provide a physical barrier to pathogens. The selection of these two cell lines was strategic, as they each represent distinct yet interrelated biological functions of the ocular surface, both of which require robust protection against environmental stressors.

The corneal limbus and conjunctiva are key epithelial tissues of the ocular surface. They not only form a physical barrier against external harmful substances but also serve as a potential entry point for nutrients from the tear film. The ocular surface is constantly exposed to environmental factors such as ultraviolet light and atmospheric pollutants, making it susceptible to oxidative stress. Therefore, ocular epithelial cells require robust cytoprotective mechanisms to combat the constant onslaught of free radicals. EGT, an important cytoprotective agent, crosses this critical barrier and completes its transport from the apical side (toward the tear film) to the basolateral side (toward the inner eye).

The human eye is a sophisticated bio-optical system, and its anterior surface plays a dual, critical role in visual function and biological protection. The anterior surface of the eye is primarily composed of the cornea, conjunctiva, and the limbus connecting the two. As the primary refractive interface of the eye, the transparency and surface integrity of the cornea are crucial for clear vision. The conjunctiva, a thin membrane covering the inner eyelid and the anterior surface of the sclera, is responsible for lubrication and protection. The corneal epithelium located on the anterior surface of the cornea and the conjunctival epithelium are two tissues with significant structural and functional differences. These three interrelated but unique anatomical units or tissues, aiming to construct a detailed “mental map” of the anterior surface of the eye through detailed descriptions of them to compensate for the limitation of not being able to directly perform image analysis.

The limbus is defined as the annular transition zone between the transparent cornea and the opaque sclera, typically 1 to 2 mm wide. On the anterior surface of the eye, the limbus is located where the corneal curvature protrudes from the sclera. Macroscopically, this region marks the transition between two distinct tissue properties: from transparent to opaque, and from avascular to vascular. This transition is not a simple linear boundary but rather a sophisticated biological interface.

The cornea's transparency stems largely from the highly ordered and uniform arrangement of collagen fibers in its stroma, along with their precise thickness and density. In contrast, the collage fibers in the sclera are arranged more randomly and interwoven, giving it an opaque white appearance. The limbus is the precise anatomical location where this structural transition occurs. This shift from highly ordered to random arrangement makes the limbus more than a simple landmark, but rather an interface optimized for both biomechanics and optical function. It maintains corneal curvature and refractive properties while smoothly transitioning to the tough, protective structure of the sclera.

The anatomy of the limbus is a multilayered complex, encompassing several key tissues from the outermost to the deepest layers. The outermost layer is the conjunctiva, which covers the surface of the limbus and extends to the inner eyelid and sclera. Beneath the conjunctiva lies a unique tissue known as the Palisades of Vogt (POV). The Palisades are a series of radially arranged fibrovascular ridges composed of blood vessels and fibrous tissue. These ridges contain depressions known as limbal crypts or niches, which are home to limbal epithelial stem cells (LESCs). The blood vessels in the Palisades of Vogt not only provide nutrients to these stem cells but also protect them from ultraviolet (UV) radiation through their unique structure. The anatomy of the Palisades of Vogt varies uniquely among individuals, earning them the nicknames “conjunctivoglyphics” or “limboglyphics.” This highly specialized microstructure suggests that the palisade of Vogt is not a simple anatomical feature but rather a carefully designed microenvironment that shelters and supports the cornea's most critical regenerative cells, highlighting its irreplaceable biological importance.

Below Vogt's palisade lies the episcleral tissue, a thin layer of loose connective tissue. Further below lies the corneal-scleral stroma junction, where the ordered collagen fibrils of the cornea meet the disordered collagen fibrils of the sclera. At the deepest part of the limbus, the collecting channels and Schlemm's canal, responsible for the outflow of aqueous humor, are located.

The functions of the limbus are diverse and crucial. First, it provides nutrition for the avascular cornea. Although the cornea itself lacks a direct vascular supply, the rich vascular system of the limbus and the tear film on the ocular surface provide essential nutrients. Second, the limbus plays a crucial barrier role. It provides a structural barrier, preventing the migration of conjunctival epithelial cells toward the transparent corneal surface. Third, the limbus facilitates the drainage of aqueous humor through its deep collecting ducts and Schlemm's canal, thereby regulating intraocular pressure and preventing pathological conditions such as glaucoma. Finally, and most crucially, the limbus serves as a regenerative source for the corneal epithelium. Limbal stem cells (LSCs) in Vogt's palisade, with their high proliferative potential and clonality, are responsible for the continuous renewal of the entire corneal epithelium, ensuring its health and transparency.

The corneal epithelium is the outermost layer of the cornea, directly overlying the iris and pupil. It is transparent layer of tissue that accounts for approximately 10% of the cornea's total thickness. As the eye's outermost layer, the corneal epithelium plays a dual role: a protective physical barrier and a key layer in maintaining a smooth refractive surface.

The corneal epithelium is a non-keratinized, stratified structure with a thickness of approximately 5 to 7 cell layers. From deep to superficial, it contains three different cell types:
(A) Basal cells: The deepest layer, columnar in shape, is connected to the underlying basement membrane by hemidesmosomes. This cell layer contains limbal epithelial stem cells (LESCs) and their progeny, transient amplifying cells.
(B) Pterygoid cells: The middle layer, polygonal in shape, usually with two to three cell layers.
(C) Squamous cells: superficial, flat, with three to four cell layers, and flattened nuclei.

The continuous renewal of the corneal epithelium is a dynamic process driven by limbal stem cells. Limbal epithelial stem cells (LESCs) divide and differentiate into transient proliferating cells, which then migrate toward the central and superficial layers. During this migration, they further differentiate into wing cells and superficial squamous cells. Ultimately, these superficial cells are shed and washed away by tear fluid. This continuous process ensures that the entire corneal epithelium is renewed every 7 to 10 days.

At the cellular level, this regenerative process involves a well-defined cell lineage. Studies have found that the generation of the corneal epithelium involves three types of clonal keratinocytes: holoclones, meroclones, and paraclones. Holoclones are true stem cells, possessing the greatest proliferative potential and capable of giving rise to the other two cell types. Meroclones and paraclones are known as transient proliferating cells, whose proliferative capacity is much lower than that of holoclones. All three types of cells are present in the basal layer of the cornea, but holoclones are the least abundant, comprising only 10% to 15%. This cellular hierarchy provides a molecular basis. For example, in clinical practice, the identification and qualification of holoclones using the biomarker p63 can effectively assess the success rate of stem cell transplantation, demonstrating a shift in diagnosis and treatment from macroscopic anatomy to microscopic cell biology.

The conjunctiva is a thin, transparent, mucous membrane that covers the inside of the eyelid and the front of the sclera. It does not cover the cornea. Anatomically, the conjunctiva can be divided into three regions:
(A) Palpebral conjunctiva: Lyings the inner surface of the eyelid.
(B) Bulbar conjunctiva: Covers the front surface of the sclera.
(C) Conjunctival fornices: Located at the junction where the palpebral and bulbar conjunctiva meet, they form a loose, sac-like structure that makes it impossible for foreign objects such as contact lenses to be “lost” to the back of the eye.

The conjunctival epithelium stem cells (CESCs) are bipotent cells, primarily located in the basal layer of the conjunctival epithelium, more specifically, scattered in the fornix and palpebral conjunctiva regions, responsible for regenerating conjunctival epithelium and secreting mucin from goblet cells. Unlike stem cells that differentiate into only one type of specialized cell, bipotent stem cells have the capacity to give rise to two distinct cell lineages. The two cell types they produce are the non-goblet conjunctival epithelial cells (also referred to as keratinocytes) and specialized goblet cells, which secrete mucus. These bipotent stem cells are essential for continuously replacing cells and maintaining the health and function of the conjunctival surface. In the event of an injury or damage to the ocular surface, these stem cells activate, proliferate, and differentiate to restore the damaged tissue. Conjunctival stem cells have a high capacity for self-renewal, ensuring a continuous supply of new cells. Their commitment to either the non-goblet or goblet cell lineage is regulated by various factors, including growth factors and surrounding cellular environment.

Like other epithelial tissues, the conjunctival epithelium stem and progenitor cells are usually located in the basal layer, closest to the basement membrane. This location is relatively protected and can easily receive nutrients and signals from the underlying matrix, allowing them to proliferate and differentiate. Unlike other stem cells, the conjunctival epithelium stem cells normally divide very slowly, remaining in a relatively quiescent state. This property protects them from the accumulation of DNA replication errors, thereby maintaining the tissue's regenerative capacity over the long term.

HCLE (human corneal-limbal epithelial) and HCjE (human conjunctival epithelial) cells are commonly used as in vitro models to study the transport of various substances, including ergothioneine, across the ocular surface. However, their kinetic properties in ergothioneine transport via OCTN1 are not identical to those of actual limbal epithelial stem cells (LESCs) and conjunctival epithelial stem cells (CESCs).

While HCLE/HCjE cells provide valuable insights, several factors can cause their kinetics to differ from the in vivo situation:
(A) Cellular Heterogeneity: In the natural eye, LESCs and CESCs exist in a complex microenvironment. They are part of a heterogeneous population of cells, each with distinctive metabolic and transport activities. HCLE/HCjE cell lines, on the other hand, are often more homogeneous, which can simplify the transport dynamics but also fail to capture the complexity of the native tissue.
(B) Cellular Differentiation: HCLE/HCjE cells are often immortalized or cultured under conditions that promote proliferation rather than full differentiation. The expression and activity of OCTN1 can change as stem cells differentiate. The kinetic parameters observed in these cell lines might not fully represent the transporters in the less differentiated stem cell population or the terminally differentiated cells they produce.
(C) Culturing Conditions: The in vitro environment, including the growth medium, substrate, and lack of systemic factors like hormones and growth factors, can influence the expression and function of OCTN1. This can lead to different kinetic values (e.g., Km and Vmax) compared to the in vivo environment.

Despite the differences, studying EGT transport in HCLE/HCjE cells is needed for understanding the basic mechanism of how this antioxidant is taken up by ocular epithelial cells. The mechanism is believed to be analogous in actual stem cells. Here is how it works:
1) OCTN1-Mediated Transport: OCTN1 is a member of the solute carrier family of transporters. It is responsible for the active, sodium-dependent transport of EGT. This process is saturable, meaning that there is a maximum rate at which EGT can be transported, which is a key characteristic of carrier-mediated transport.
2) Role of EGT: EGT is a potent antioxidant that protects cells from oxidative stress. LESCs and CESCs are highly vulnerable to oxidative damage due to environmental exposure (e.g., UV radiation). The active uptake of EGT via OCTN1 is thought to be a critical protective mechanism for these stem cells. It helps maintain their viability and stemness, which is essential for the continuous regeneration of the corneal and conjunctival epithelia.
3) Analogous Mechanism: The kinetic studies in HCLE/HCjE cells provide a fundamental understanding of the OCTN1 transporter's affinity for EGT (Km) and its maximum transport capacity (Vmax). These parameters, while potentially different in magnitude, likely reflect the same underlying transport mechanism as in the native stem cells. The knowledge gained from these models can be used to predict how external factors or drugs might influence EGT uptake.

HCLE (human corneal-limbal epithelial) and HCjE (human conjunctival epithelial) cells are commonly used as in vitro models to study the transport of various substances, including EGT, across the ocular surface. However, their kinetic properties in EGT transport via OCTN1 are not identical to those of actual limbal epithelial stem cells (LESCs) and conjunctival epithelial stem cells (CESCs).

While HCLE/HCjE cells provide valuable insights, following several factors can cause their kinetics to differ from the in vivo situation:
1) Cellular heterogeneity: In the natural eye, LESCs and CESCs exist in a complex microenvironment. They are part of a heterogeneous population of cells, each with distinct metabolic and transport activities. HCLE/HCjE cell lines, on the other hand, are often more homogeneous, which not only can simplify the transport dynamics but also fail to capture the complexity of the native tissue.
2) Cellular differentiation: HCLE/HCjE cells are often immortalized or cultured under conditions that promote proliferation rather than full differentiation. The expression and activity of OCTN1 can change as stem cells differentiate. The kinetic parameters observed in these cell lines might not fully represent the transport in the less differentiated stem cell population or the terminally differentiated cells they produce.
3) Culturing conditions: The in vitro environment, including the growth medium, substrate, and lack of systemic factors, can influence the expression and function of OCTN1. This can lead to different kinetic values (e.g., KM and Vmax) compared to the in vivo environment.

Despite the differences, studying EGT transport in HCLE/HCjE cells is crucial for understanding the basic mechanism of how this antioxidant is uptaken by ocular epithelial cells. The mechanism is believed to be analogous in the stem cells.

The mechanism operates as follows:
1) OCTN1-mediated transport: OCTN1 is a member of the solute carrier family of transporters. It is responsible for the active sodium-dependent transport of EGT. This process is saturable, meaning that there is a maximum rate at which EGT can be transported, which is a key characteristic of carrier-mediated transport.
2) Role of EGT: EGT is a potent antioxidant that protects cells from oxidative stress. LESCs and CESCs are highly vulnerable to oxidative damage due to environmental exposure (e.g., UV radiation). The active uptake mechanism of EGT via OCTN1 is thought to be a critical protective mechanism for these stem cells. It preserves their viability and stemness, which is crucial for ongoing regeneration of corneal and conjunctival epithelium.
3) Analogous mechanism: The kinetic studies in HCLE/HCjE cells provide fundamental guidance for the kinetics and mechanisms in LESCs/CESCs cells.

It is not just about absorption. For example, whether it accumulates within cells or is transported to deeper tissues to exert its effects. Whether OCTN1 participates in bidirectional transport or whether other proteins assist.

The polarity of epithelial cells and the different protein distributions on the apical and basal membranes. For example, GLUT1 is located on the basal membrane, while OCTN1 is enriched in or located on the apical membrane, indicating a clear division of labor. Whether EGT can enter through the apical membrane and exit from the basal membrane, reaching the intraocular tissues.

The distinction between absorption and transport should be clearly explained, emphasizing that OCTN1 is primarily responsible for uptake, while other mechanisms may be involved on the basolateral side, such as diffusion or different transporters. Also, possible metabolism or accumulation should be mentioned, which is useful in certain aspects of the present invention, such as whether residence time or co-transport should be considered.

OCTN1 is enriched in the apical membrane of corneal and limbal epithelial cells (the side facing the tear fluid). Its physiological role is very clear: it captures EGT from the tear fluid and internalizes it. This is an active or facilitated diffusion transport process, unidirectional from the outside to the inside of the cell.

OCTN1 is the main transporter that brings EGT into cells. It is highly expressed in tissues with high oxidative stress, like red blood cells, monocytes, and certain parts of the eye, which is why it is so important for the initial uptake at the apical membrane.

The existence of a dedicated high-affinity transporter like OCTN1 suggests that EGT is not just a general antioxidant, but a critically important one that the body actively seeks to acquire and accumulate in vulnerable tissues.

While the specific transport for basolateral movement is not known, research has shown that EGT is effectively transported to and accumulated in the retina, particularly in the macula. This highlights a mechanism for getting the compound across the blood-retinal barrier (BRB) to protect these vital cells from oxidative damage.

While OCTN1 is recognized as a transporter for EGT, the precise mechanism by which it facilitates basolateral movement from the epithelium to the inner eye tissues, including the retina and macula, is still under investigation.

The high expression of OCTN1 in single (monocytes) or isolated cells is primarily related to its core functions and the unique physiological stresses faced by single cells. This can be understood from the following key points:
1) OCTN1's core function: Transporting EGT. In recent years, OCTN1's most important function has been identified as a high-affinity transporter for EGT. EGT is a very powerful natural antioxidant that the human body cannot synthesize on its own and must be obtained from the diet (such as mushrooms and grains).
2) Powerful cell protection: EGT effectively neutralizes various reactive oxygen species (ROS) and free radicals, protecting DNA, mitochondria, proteins, and lipids within cells from oxidative damage. Unlike other antioxidants like glutathione, EGT dose not undergo auto-oxidation in the body and is extremely stable, earning it the nickname “the longevity vitamin” and “the ultimate antioxidant”.

When activated to execute their antibacterial functions, immune cells—including macrophages, monocytes, leukocytes, erythrocytes, and neutrophils—generate significant quantities of reactive oxygen species (ROS) as part of their defense mechanisms. While this process can kill pathogens, it also places significant oxidative stress on the immune cells themselves. These cells are typically “roaming” and relatively independent within the body.

Cells in tissues can share nutrients and signaling molecules through a “bystander effects.” However, an isolated single cell must be self-reliant in building and maintaining its own robust defense system, as it cannot easily rely on help from neighboring cells. That is why single cells need to express high levels of OCTN1. To survive in isolation, single cells need to maximize their antioxidant defenses. High OCTN1 expression allows them to more efficiently absorb EGT, the “ultimate antioxidant”, from the outside world, building a powerful and independent “personal defense system.”

Single cells (such as circulating immune cells) are like soldiers on a mission in the field, directly exposed to danger. Therefore, they need to carry the most sophisticated personal protective equipment (high levels of EGT), and OCTN1 is their “dedicated channel” for acquiring and donning this gear. High OCTN1 expression means they can equip themselves more quickly, improving their survival and combat effectiveness in harsh environments.

OCTN1 is highly expressed in bone marrow, spleen, and various immune cells or T lymphocytes. These cells are typical examples of cells that circulate and migrate throughout the body and respond to the intense oxidative stress generated during inflammation and infection.

At the site of inflammation, oxidative stress levels in the environment rise dramatically. Immune cells infiltrating these areas upregulate OCTN1 expression to increase their uptake of EGT, thereby protecting themselves from damage and maintaining healthy immune function.

Some studies have found that isolated and cultured stem cells (a single-cell state in vitro) also highly express OCTN1, which may help maintain the genomic stability and “stemness” of stem cells and prevent differentiation or aging caused by oxidative damage.

EGT needs to go from the tear side (apical membrane) to the aqueous humor or the deep corneal tissue (basolateral membrane side). This process requires multiple steps and possibly different transport proteins to work together.

Once EGT transported from the tear into the cytoplasm via OCTN1 on the apical membrane, a significant portion remains inside due to its potent antioxidant properties, playing a key role in protecting cells from oxidative damage. The intracellular accumulation of EGT is crucial for ocular tissues such as cornea and lens, which are exposed to high oxygen levels and photooxidative stress.

Basolateral membrane efflux of EGT may be mediated by other transporters. OCTN1 is likely not solely responsible for exporting EGT across the cell. This process may be achieved through the following means:
1) Simple diffusion: If the intracellular concentration is high enough, some EGT molecules may diffuse out passively.
2) Other basolateral membrane transport protein: More likely, cells, other types of transporters exist on the basal membrane of cell, responsible for transporting EGT from the intracellular and extracellular space to the basolateral interstitial space. For example, some multidrug resistance proteins (MRPs) or organic anion transporters (OATs) may be involved in this process, but detailed research in this area is still ongoing.

In summary, while HCLE/HCjE cell models are simplified systems, they serve as a powerful tool to study the fundamental kinetics and mechanism of OCTN1 mediated EGT transport. The findings from these models are generally considered representative of the underlying biological process, even if the specific numerical values for kinetic parameters might not perfectly match those of native LESCs and CESCs due to differences in cellular environment and heterogeneity, and cell health in the eye.

The extensive research results on carnitine transport through OCTN1/2 in the HCLE/HCjE cell model provide valuable guidance for future studies on the transport kinetics of EGT via OCTN1. These research results not only establish reliable research methods but also allow us to conduct specific and effective research on the transport characteristics of EGT. The study of carnitine transport provides a mature and reusable scientific framework for the study of EGT, which is mainly reflected in the following aspects:
1) Model Validation and Applicability: HCLE and HCjE cell lines have been shown to be excellent in vitro models for nutrient transport in the eye, particularly the cornea. Carnitine studies have demonstrated that these cell lines endogenously express the OCTN1/2 transporter, whose function closely correlates in vivo conditions. This eliminates the need for model selection and validation in EGT research, allowing direct use of these cell lines for experiments.
2) Maturity of experimental methodology: Carnitine research has established a standard transport experimental protocol, including:
   (a) Uptake kinetics curve: By measuring the intracellular carnitine uptake at different time points, the stable period of transport is determined, providing a time basis for subsequent experiments.
   (b) Concentration-dependent uptake: The transport rate is measured at different substrate concentrations to obtain transport kinetic data.
   (c) Inhibitor and competition experiments: Use Known OCTN1/2 inhibitors (e.g., MCT, quinidine) or competitive substrates (e.g., high-dose unlabeled carnitine or EGT) to verify the specificity and saturability of transport.
      These methods can be directly applied to EGT to ensure the reliability of the results.
3) Template for kinetic analysis: carnitine transport studies typically use the Michaelis-Menten kinetic model to analyze data and calculate two key parameters: affinity (Km) and maximum transport rate (Vmax), where km reflects the affinity of the transporter to the substrate (the smaller the value, the higher the affinity); Vmax reflects the maximum transport capacity per unit time (the higher the value, the faster the transport speed).

This mature mathematical model can be directly used to analyze the transport data of EGT, thereby accurately quantifying its transport efficiency.

Based on an understanding of carnitine and EGT as substrates of OCTN1, aspects of the present invention can provide some predictions about the transport kinetics and transport velocity of EGT.

The following specific inferences are considered:
(A) Influence of affinity (Km): Although both OCTN1 and OCTN2 can transport carnitine, OCTN1 is considered the primary physiological transport of EGT. OCTN1 is considered the primary physiological transporter of EGT. From an evolutionary and physiological perspective, transporters generally have a higher affinity for their primary physiological substrate. Therefore, it can be inferred that:
a) The affinity (Km) of EGT for OCTN1 is likely significantly lower than the affinity (Km) of carnitine for OCTN1.
b) This means that at physiological concentrations OCTN1 can bind and transport EGT more efficiently and preferentially. This is most important for predicting EGT can transport very fast through ocular surface to retina via OCTN1 from the analysis of existing experimental results of carnitine transport.
(B) Inference of transport rate: The transport rate at low substrate concentrations is mainly determined by the affinity (Km). Since EGT likely has a higher affinity for OCTN1 (lower Km value), we can infer that in the corneal environment, when both EGT and carnitine are at their respective physiological concentrations (usually low), the uptake rate of EGT through OCTN1 will be faster than the uptake rate of carnitine through OCTN1. This suggests that the more important function of OCTN1 in the eye is to efficiently absorb and accumulate EGT to exert its powerful antioxidant and cell-protective effects, while carnitine may mainly rely on OCTN2 to maintain its function.

In summary, studies on carnitine transport in HCLE/HCjE cells provide a solid methodological foundation for the study of EGT, including experimental design, model selection, and data analysis. Based on this foundation, aspects of the present invention can specifically infer that EGT, as the primary physiological substrate of OCTN1, most likely has a higher affinity (lower Km value) for this transporter, resulting in a faster transport rate than carnitine under physiological conditions. This is highly consistent with its physiological function as an important antioxidant in the eye.

The transport mechanism of EGT in HCLE/HCjE cells via OCTN1 is essentially the same as that in other cell lines (such as HEK293 cells), both of which are dependent on OCTN1 and are secondary active transporters. The core mechanism is as follows:
1) Sodium ion cotransport (Na+-cotransport): This is the key driving force of transport. Cells use the energy generated by the sodium ion concentration gradient inside and outside the cell membrane to transport a sodium ion and an EGT molecule from outside the cell to inside the cell at the same time. This process does not require direct consumption of ATP but rather utilizes the sodium ion gradient maintained by the sodium-potassium pump (Na+/K+ pump) on the cell membrane.
2) Saturation: HCLE/HCjE cells have a limited amount of OCTN1 transporter proteins. When the extracellular concentration of EGT increases to a certain level, all transporter proteins are occupied, and the transport rate reaches its maximum, no longer increasing with increasing concentration. This demonstrates that this transport process is mediated by a specific protein.
3) High specific: OCTN1 is highly selective for its substrates. While carnitine and other organic cations are known to bind to OCTN1, it is believed that EGT is OCTN1's natural substrate, with which it has the highest affinity.

EGT transports in HCLE/HCjE cells via OCTN1 is a Na+-dependent secondary active transport. This enables these important ocular surface cells (most likely apical membrane epithelium) to efficiently take up EGT, adapting to their unique physiological environment and providing essential protection against oxidative stress.

In summary, EGT transport via OCTN1 is a highly specific and multifactorial process that ensures that cells can effectively intake this important antioxidant to exert its protective effects.

### Transport of EGT Through Ocular Surface

In any epithelial tissue, whether from the limbus or the conjunctiva, two cell polarities, apical and basolateral, can be distinguished.

The outer surface of epithelium is apical membrane. Apical cells refer to the cells facing the tissue cavity or external environment. This part of the cells typically contains structures such as microvilli or cilia, which perform functions such as absorption, secretion, and protection.

The inner epithelium is basolateral membrane. Basolateral cells refer to the cells facing the basement membrane and underlying connective tissue. These cells are primarily responsible for adhesion, signaling, and exchanging substances with neighboring cells and the extracellular matrix (ECM).

The limbus is the transitional area connecting transparent cornea and the opaque conjunctiva, which contains abundant stem cells in the innermost layer adjacent to the basement membrane. The limbal stem cells have properties allowing them to divide, differentiate, and replenish the overlying epithelium.

The conjunctiva is the mucous membrane that covers the inside of the eyelid and part of the surface of the eyeball.

Apical cells in the conjunctival epithelium, which is in the outermost layer of the epithelium coming into direct contact with tear fluid, contain goblet cells that secrete mucins, which are important components of the tear membrane and help maintain moisture on the surface of the eye.

Regarding the anatomical location, the conjunctival stem cells are mainly located in the crypts and folds of the fornix, and behind the gray line of the eyelid margin. These locations provide a good protective environment and rich blood supply for the stem cells.

In terms of function, conjunctival stem cells are mainly responsible for maintaining the renewal and repair of the conjunctival epithelium, including the production of goblet cells, which are essential for the stability of the tear film and the health of ocular surface by secreting mucins.

While limbal stem cells (LSCs) and conjunctival stem cells (CSCs) are anatomically and functionally distinct, they work together to maintain ocular surface health, and sometimes in pathological situations such as limbal stem cell deficiency (LSCD), the conjunctival epithelium may grow toward the cornea.

Anatomical location of conjunctival epithelial stem cells, the distribution of conjunctival stem cells is not as clearly defined as that of limbal epithelial stem cells in the Vogt palisade aera, but may reside in the following different anatomical locations:
1) Fornix: The conjunctival fornix is the deepest part of the conjunctival sac and is divided into the superior fornix, inferior fornix, and lateral fornix. Its numerous wrinkles and dense blood vessels offer a nutrient-rich, protective niche for stem cells. Studies indicate that the fornix has high cell proliferation and is a primary site for conjunctival epithelial stem cells.
2) Mucocutaneous junction: The skin-mucosal junction, also known as the area near the opening of the meibomian glands, serves as a transitional zone where cells with stem cell properties may also exist.
3) Crypts and folds: The conjunctiva's folds and crypts also contribute to increased surface area and provide potential habitats for stem cells.

The function of LESCs is regulated by their environment, called the stem cell niche, which includes cells and molecules that maintain their activity and differentiation. Traditionally, LESCs were thought to be rare, slow-cycling cells, but newer research suggests there may be different populations with varying roles.

Like LESCs, CESCs divide very slowly under normal circumstances and are in a relatively static (or quiescent) state. This property protects them from the accumulation of DNA replication errors, thereby maintaining the tissue's regenerative capacity over the long term.

Conjunctival stem cells are not evenly distributed throughout the conjunctival epithelium. Studies have shown that the area with the highest stem cell activity are mainly distributed in fornix (The “crease” area between the eyelid and the eyeball. This area is thought to be rich in stem cells, possibly because it is better physically protected.) and palpebral conjunctiva (the part covering the inner surface of the eyelid). In contrast, the number and activity of stem cells in the basal layer of the bulbar conjunctiva (mucocutaneous junction), which covers the front of the eyeball, are much lower.

The functions of these conjunctival stem cells are as follows:
1) Self-renewal: after division, one daughter cell retains the stem cell characteristics, while the other enters the division pathway.
2) Producing progenitor cells: Progenitor cells have stronger proliferation ability, but the direction of differentiation is determined.
3) Differentiation: The cells eventually differentiate into various mature cell types in the conjunctival epithelium, including but not limited to, Goblet cells (secrete mucin, a key component of a healthy tear film) and non-goblet epithelial cells.

The limbal corneal epithelium and conjunctival epithelium are the key membrane structures regulating the entry of drugs from tears into the anterior chamber. Studies have demonstrated that OCTN1's mRNA and protein expressions have been detected in human corneal and conjunctival epithelial cells (HCLE and HCjE). Within these epithelial cells, OCTN1 is primarily localized to the apical membrane. This polar localization is crucial for ocular pharmacokinetics. OCTN1, when localized apically, facilitates the uptake of EGT from tears or lacrimal fluid and transports it into corneal epithelial, limbal epithelial, and conjunctival epithelial cells.

The cornea is the transparent, avascular tissue at the front of the eye. At the cornea, EGT transports from corneal epithelial cells to the Bowman's layer, stroma, and anterior chamber.

The Bowman's layer in the middle of cornea is essentially a static anatomical barrier and doesn't actively transport itself. It is essential to describe the mechanism by which EGT passively diffuses across the barrier and how active transport mechanisms employed by epithelial and endothelial cells establish the necessary driving force. The stromal layer makes up most of the volume, with its diffusion efficiency mainly determined by tissue resistance, ionic strength, and fixed charge density. The power source is indeed the core. It is necessary to clearly point out that OCTN1-mediated active absorption is the “engine” that starts the entire process. Although subsequent diffusion is passive, the establishment of the concentration gradient is completely dependent on this initial active step. The metaphor of “pumped by a pump (active transport), flow in a pipe, and absorbed by a sponge” describes the mechanism of the transporting power source.

The limbus is the transition zone between the cornea and the sclera (white of the eye), a ring-shaped band approximately 1.5-2.0 mm wide. The limbus contains the habitat of limbal stem cells, which are responsible for the continuous renewal of the corneal epithelium. It is also the main channel (conventional pathway) for aqueous humor to drain out (trabecular meshwork). At the limbus, EGT transports from limbal epithelial cells to limbal corneal stroma, Descent's membrane, limbal corneal endothelial cells, basal mask and aqueous humor.

The apical membrane of limbal epithelium is the outermost layer of the epithelial cells, its surface covering with microvilli and microfolds, which help maintain the stability of the tear film. At the limbus, this surface contacts the tear film, comprising approximately 10-15% of the total ocular surface.

EGT moves across tight junctions and interstitial spaces between epithelial cells. The tight junctions of the limbal epithelium are not as tight as those of the central corneal epithelium, which provides a potential channel for aqueous humor outflow. EGT passes directly across epithelial cells by active transport or passive diffusion.

At the limbus, the Descemet's membrane of the cornea terminates. The Descemet's membrane is the most specialized part of the corneal stroma. It is not a true membrane, but a layer of acellular collagen fibers. Therefore, at the limbus, directly beneath the epithelial basement membrane is the edge of the corneal stroma, whose structure begins to become similar to that of the sclera, with a more irregular arrangement of collagen fibers.

After passing the entire matrix, it reaches the Descemet's membrane. The Descemet's membrane is the basement membrane of the corneal endothelial cells, but it is very tough and elastic. It is composed of type IV collagen and other proteins. The inner side of the Descemet's membrane is a single layer of corneal endothelial cells. Endothelial cells are the final cellular barrier between the aqueous humor and the corneal stroma. These cells are separated by tight junctions and gap junctions. However, these tight junctions are “leaky”, allowing water and ions to pass through but preventing large proteins from freely passing through.

The main function of endothelial cells is to actively pump out excess water that has infiltrated the stroma to maintain corneal dehydration and transparency. This “pump” is located on the lateral membrane of the endothelial cells.

The basal surface of the endothelial cell (i.e., its bottom plasma membrane) is anchored by its own basement membrane (i.e., Descemet's membrane forms part of the basal surface, which is directly adjacent to the aqueous humor at the plasma membrane interface. Therefore, the basal membrane of the corneal endothelial cells is the true interface between the aqueous humor and the eye tissues in anatomy.

The conjunctiva is a thin, transparent membrane covering the inside of the eyelid (palpebral conjunctiva) and the front of the sclera (bulbar conjunctiva). EGT moves from epithelial cells to the lamina propria, then from the conjunctiva into the sclera, ciliary body, and iris. It may also spread laterally to the limbus and eventually reach the aqueous humor. To access the eye's interior, EGT must traverse these layers.

The outermost layer of the conjunctival epithelium covering the sclera (bulbar conjunctiva) or tarsal plate (palpebral conjunctiva). The apical membrane is oriented toward the tear film, with microvilli on its surface that play a role in stabilizing the tear film and anchoring mucin. This marks the point where the pathway meets the tear fluid and external environment.

The conjunctival epithelium usually has 2-5 layers of cells (stratified columnar epithelium containing goblet cells). EGT must transport through the epithelium via either the transcellular pathway (directly across cells) or the paracellular pathway (through intercellular junctions). The tight junctions of the conjunctival epithelium are relatively loose, allowing some large molecules to pass through, which is an important pathway for ocular drug absorption.

After transporting through the epithelium, EGT reaches the basement membrane and then enters the conjunctival lamina propria. The lamina propria contains blood vessels, lymphatic vessels, nerves, and immune cells (such as lymphoid follicles, especially in the fornix and palpebral conjunctiva). Many drugs (such as ingredients in the eye drops) are absorbed into the systemic circulation through conjunctival vessels, which is a major “loss route”.

Beneath the conjunctiva, there is a thin, tough membrane of fibrous tissue called Tenon's capsule (fascia of the eye), which is loosely connected to the surface of the sclera. The sclera is composed of dense type 1 collagen fibers, which is very strong and serves as the main barrier to protect the contents of the eye.

Behind the limbus, there are pathways through which the anterior ciliary artery and nerve enter the eyeball. Theoretically, very small particles could enter through the spaces around these vessels. Some drugs (such as prostaglandin analogs) transport the ciliary body region by diffusion across the conjunctiva and sclera in the corneoscleral limbus region.

After passing through the sclera, EGT reaches the ciliary body. The ciliary body consists of several parts:
1) Ciliary muscle: Smooth muscle responsible for adjusting focal length.
2) Ciliary process: This is the key to the path. The ciliary process is covered by a rich capillary network and non-pigmented epithelial cells/pigmented epithelial cells.
3) Aqueous humor: Aqueous humor is actively secreted by the non-pigmented epithelial cells of the ciliary process.

To cross the ciliary epithelium, EGT must exit the blood vessels in the ciliary body and pass through two cell layers:
1) Ciliary pigment epithelium,
2) Non-pigmented ciliary epithelium.

The lateral and apical membranes of the non-pigmented epithelial cells are covered with pumps and channels that actively pump ions and water from the blood into the intraocular cavity to form aqueous humor.

Once EGT successfully passes through the ciliary non-pigmented epithelium, it enters the posterior chamber of the eyeball.

This “indirect” or “circle around the eyeball” pathway helps explain how certain eye drops work, particularly those affecting muscle contraction and relaxation. For example, prostaglandins and analogs, which lower intraocular pressure, are absorbed through the conjunctiva, then penetrate the sclera and act on the ciliary body and uveoscleral pathways to increase aqueous humor outflow. They may not necessarily need to enter the anterior chamber.

Studies show the expression of OCTN1 and OCTN2 in human corneal limbal epithelial (HCLE) cells and human conjunctival epithelial (HCjE) cells. OCTN1 was mainly observed at the apical membrane of these ocular epithelial cells. Although the experiments used carnitine as a surrogate substrate, and given that OCTN1 typically transports EGT, these results indicate that EGT transport is likely mediated by OCTN1 at the apical membrane of the ocular epithelium.

The OCTN1 transporter located on the apical membrane utilizes the sodium ion electrochemical gradient to actively translocate EGT from the tear film into the cytoplasm. The targeted localization of OCTN1 within the apical membranes of conjunctival and limbal epithelial cells indicates a purposeful physiological defense mechanism. The ocular surface, being continually exposed to external elements such as ultraviolet radiation and atmospheric pollutants, encounters significant levels of reactive oxygen species (ROS). By placing the EGT transporter at the outermost cellular boundary, the body enables rapid accumulation of EGT, which serves as an immediate defense against oxidative stress. This system exemplifies both efficiency and specificity, as EGT uptake occurs directly at the interface with the environment, circumventing the need to cross multiple cellular barriers. Such localization is intrinsically related to the overarching protective role of ocular epithelial cells.

### Kinetic Analysis of Apical Uptake

Since EGT does not easily cross cell membranes, OCTN1 on the apical membrane of corneal, limbal, and conjunctival epithelial cells enables quick and efficient uptake. This process involves active carrier-mediated transport, ensures the bioavailability of EGT and effectively circumvents the permeability restrictions of hydrophilic molecules imposed by the tight junctions of the corneal epithelium, offering significant advantages for topical drug delivery.

The cornea is the primary physical barrier to ocular absorption. Positioning the highly efficient transporter OCTN1 or ETT at the apical surface of the corneal epithelium ensures maximum capture and initial retention of any EGT that may be present in the tear film, whether from dietary sources or topically applied. This setup efficiently ensures quick and sustained levels of the key antioxidant at the front of the eye.

While studies have confirmed that OCTN1 mediates apical membrane uptake, EGT must then efficiently cross the basolateral membrane of the epithelium to enter the stroma and aqueous humor. Although the transporters responsible for basolateral efflux have not been clearly identified, net influx is essential to maintain EGT supply to the anterior chamber. OCTN1 and OCTN2, key L-carnitine transporters, are found together in the corneal epithelium, highlighting a coordinated system for regulating osmoprotectants and thione antioxidants in the eye's anterior segment.

OCTN1, first cloned from fetal liver in 1997, is a potent and highly efficient EGT transporter. EGT is extremely hydrophilic, with a high affinity in the physiological pH range. It cannot penetrate cell membranes without help from other transporters, mainly OCTN1. OCTN1 can transport EGT into cells with extremely high efficiency. The discovery of the EGT transporter OCTN1 created strong expectations for the beneficial effects of EGT. OCTN1 is currently the sole biomarker for EGT activity; cells with high OCTN expression efficiently accumulate EGT and show strong transporter activity. OCTN1 enables EGT absorption from food, eye drops, and injections, as well as its redistribution and reabsorption in the kidneys and eye tissues. High OCTN1 expression was closely linked to elevated EGT levels in established ocular transport data.

Recent Michaelis-Menten kinetic analyses of EGT transport via OCTN1 in cultured cells, including HEK293 and HCLE/HCjE cells—have demonstrated that EGT is capable of rapidly traversing the cornea-limbus-conjunctiva surface of the eye and diffusing into various ocular regions, including the retina. The Km and Vmax of EGT transport via OCTN1 in model cells were 10-25 μM and 25-50 μL mg⁻¹ (protein) min⁻¹, respectively.

EGT may enter erythrocytes via the OCTN1 transporter and diffuse into the aqueous humor of both the anterior and posterior chambers, exiting through the TM-SC outflow and uveoscleral pathways. Its rapid transport in the ophthalmic artery and vein suggests potential as a drug for reducing IOP and treating glaucoma.

EGT is a hydrophilic zwitterionic compound that is virtually impermeable to plasma membranes. Therefore, without the mediation of OCTN1, EGT cannot achieve effective intracellular accumulation. OCTN1 is a powerful and highly specific transporter specifically responsible for the uptake of EGT. Research shows EGT transport efficiency can reach 195 μL·mg⁻¹(protein)·min⁻¹. This efficiency is more than 100 times higher than that of previously considered substrates, such as carnitine or tetraethylammonium (TEA).

This high-affinity, high-efficiency transport mechanism dictates that the distribution pathway of EGT in the eye is fundamentally transporter-driven and transporter-restricted. The molecular properties of EGT result in extremely low passive diffusion efficiency, meaning that achieving effective physiological or therapeutic concentrations of EGT in ocular structures depends on the expression and activity of OCTN1 in barrier tissues. The absorption, distribution, and intracellular retention of EGT are entirely dependent on the function of this transporter. Therefore, understanding the precise expression location of OCTN1 in the eye is crucial for understanding the ocular pharmacokinetics of EGT.

To better understand EGT transport from the ocular surface to the retina via OCTN1, research on L-carnitine transport in HCLE and HCjE cell lines can clarify similar mechanisms and kinetics. However, the core findings and key insights in the flawed premise underlying the direct application of L-carnitine kinetic data in the HCLE/HCjE cell models to infer EGT transport. These basic differences prevent direct extrapolation from the initial inquiry.

L-carnitine uptake in these specific ocular cells is primarily mediated by the OCTN2 transporter, while EGT is recognized as a specific and efficient substrate for OCTN1. The analyzing of existing data and establishing of a new, more scientifically rigorous analytical framework can be done by further similar experimental studies. The targeted delivery of EGT from the ocular surface to the retina is highly plausible, but this is not based on kinetic models of L-carnitine and instead relies on unique, efficient process mediated by its proprietary transporter, OCTN1.

Findings, according to aspects of the present invention can be summarized as follows:
1) The HCLE/HCjE cell model is an effective tool for studying human ocular surface transport, but its application must be targeted to specific substrates and transporters.
2) Kinetic studies have confirmed that L-carnitine transport in HCLE and HCjE cells is a carrier-mediated process involving both high-affinity and low-affinity systems. However, antibody inhibition studies have clearly demonstrated that this transport activity is primarily attributable to OCTN2, not OCTN1.
3) In stark contrast, EGT is a highly efficient substrate for OCTN1, with its transport efficiency over 100-fold higher than that of L-carnitine.
4) OCTN1 is expressed in multiple layers of both the cornea (entry point) and the retina (target tissue). This provides a direct, biologically based, targeted delivery pathway for EGT from the ocular surface to the retina.

Research on L-carnitine transport in HCLE/HCjE cells identified kinetic parameters that contributed to the understanding of OCTN1/2-mediated transport mechanisms. These studies demonstrated that L-carnitine uptake is saturable and time-dependent, suggesting a carrier-mediated transport mechanism. Eadie-Hofstee analysis revealed two independent components of L-carnitine transport: a high-affinity system and a low-affinity system. In the Eadie-Hofstee plot, these dynamics are shown as two separate straight lines with distinct slopes and Y-intercepts.

The transport of EGT is almost entirely dominated by OCTN1, a high-affinity, high-efficiency system. The transport of EGT via OCTN2 is almost zero. The graph shows that because EGT is transported at a significant rate by only one transporter (OCTN1), its transport kinetics follow a single Michaelis-Menten equation at both low and high concentrations. On the Eadie-Hofstee plot, this appears as a single straight line, which corresponds to a low Km value. EGT showed only one system due to its reliance on OCTN1, but Eadie-Hofstee graphs used varying substrate concentrations to separate the roles of OCTN2 (primary transporter) and OCTN1 (secondary transporter) for L-carnitine into two distinct kinetic parameters.

To identify the specific transporter responsible for L-carnitine transport, a key antibody inhibition experiment was conducted. This experiment clearly confirmed the mechanism's response. The results showed that anti-OCTN2 antibodies significantly blocked L-carnitine uptake in HCLE/HCjE cells, whereas anti-OCTN1 antibodies did not. This finding is crucial. It directly demonstrated that L-carnitine transport in HCLE/HCjE cells is primarily mediated by OCTN2, with OCTN1 playing no significant role.

Studies of the OCTN1 transporter have revealed its exceptionally high specificity for EGT. A groundbreaking study identified OCTN1 (since functionally renamed ETT, for EGT transporter) as a specific transporter of EGT. Research showed that EGT has a Km of 10-25 μM for OCTN1 transport in HEK293 cells expressing OCTN1.

The study compared the transport efficiency of EGT and L-carnitine under OCTN1, finding that EGT exhibited over 100 times greater transport efficiency than L-carnitine and tetraethylammonium (TEA). This significant difference in efficiency fully demonstrates the specific selection and superior transport capacity of OCTN1 for EGT. In contrast, OCTN2, a specific transporter for L-carnitine, does not transport EGT at all.

Research shows that OCTN1 plays a key role in EGT transport within the eye. Because of its high selectivity and efficiency, EGT delivery via OCTN1 differs significantly from L-carnitine transport by OCTN2, making direct comparisons between the two processes unreliable.

To infer the ability of EGT to be transported from the ocular surface to the retina, in addition to demonstrating the molecular mechanism of its transport, it is also necessary to confirm the presence of its transport protein, OCTN1, in relevant tissues. Studies have shown that OCTN1 is expressed in multiple ocular tissues, providing a solid anatomical basis for the targeted transport of EGT.

OCTN1 is found in multiple sites throughout the eye, including the anterior and posterior chambers. In the anterior chamber, OCTN1 is expressed in the cornea, lens, and ciliary body. In the posterior chamber, OCTN1 is localized in the retina (both central and peripheral regions) and the retinal pigment epithelium (RPE)/choroid. More specific intraretinal localization reveals OCTN1 in multiple retinal layers, including the nerve fiber layer (NFL), ganglion cell layer (GCL), inner plexiform layer (IPL), inner nuclear layer (INL), and outer nuclear layer (ONL). This continuous expression pattern from the cornea to retina outlines a clear, vector-mediated transport pathway.

Based on the continuous expression pattern of OCTN1 from anterior to posterior in ocular tissues, a plausible targeted transport hypothesis can be proposed. Following topical application to the ocular surface, EGT is first uptake by the OCTN1 transporter within apical membrane and actively accumulated in the retinal layers, where it exerts its antioxidant and cytoprotective effects. This transport is not simply passive diffusion, but an active process mediated by efficient transporters. The rate of transport is not solely dependent on concentration gradients or permeability of physical barriers but is directly related to OCTN1's high affinity (low Km) and high catalytic activity (high Vmax) for EGT. OCTN1 expression in the macula further suggests that this accumulation is not random but rather has a high physiological importance, protecting the area of the eye most vulnerable to oxidative stress.

### Conventional (TM-SC) and Uveoscleral Outflow Pathways

The conventional outflow pathway is situated within the limbus region. The aqueous humor follows a standard route, beginning at the trabecular meshwork (TM) in the anterior chamber angle, subsequently passing into Schlemm's canal (SC), a circular channel encircling the cornea and embedded within the scleral tissue.

The trabecular meshwork is a porous, sponge-like tissue located at the cornea-sclera junction just beneath or on the innermost surface of the limbus. The anterior end of the Trabecular meshwork (TM) attaches to the end of Descemet's membrane, slightly anterior to the scleral process. This point of attachment is called Schwalbe's line. The posterior end of TM directly inserts into and overlies the scleral protrusion or protuberance. This is the key area where Schlemm's canal (SC), the main channel for aqueous humor to exit or flow out the eyeball, is located.

The trabecular meshwork itself can be divided into three main parts:
1) Uveal reticularis: The most inner part faces the anterior chamber, with larger pores.
2) Corneoscleral mesh: The middle part has finer mesh.
3) Juxtubular tissue: The outmost part, adjacent to Schlemm's canal, which is where the resistance to aqueous humor outflow is the greatest.

TM is a wedge-shaped meshwork that fills the inner sulcus (or groove) at (or of) the corneal-scleral junction (endoscleral groove). Its anterior boundary is the end (or terminus) of the long-bore Descemet's membrane (Schwalbe's line), and its posterior boundary is the sclera.

The trabecular meshwork sits on the scleral groove, while the scleral spur anchors it and affects its tension, influencing resistance to aqueous humor outflow—a key factor in open-angle glaucoma. The ciliary muscle adjusts pore size by pulling the scleral process, explaining dynamic drainage and drug mechanisms. The ciliary band, visible during gonioscopy, serves as a landmark to distinguish between open- and closed-angle glaucoma.

Scleral groove is located medially to the limbus, with the limbus ending in front and the scleral process behind. This groove provides structural space for the trabecular meshwork.

When aqueous humor flows out of the anterior chamber, it passes through: Anterior chamber - - - porous network of TM - - - proximal tubular tissue - - - SC - - - collecting duct - - - aqueous humor vein - - - finally merges into the blood circulation.

TM functions as a critical “filter” or “drain” in front of the scleral process and covering the inner side of the main channel for aqueous outflow (SC). Its exact location extends from Schwalbe's line to the scleral process, while the ciliary body band is the structure located behind it.

A Schlemm's canal cross-section is a flattened, annular (ring-shaped) elliptical lumen. It is a unique vascular structure that encircles the cornea at the corneoscleral junction, forming a drainage pathway for aqueous humor to exit the eye. The canal has an inner and outer wall, each lined with a single layer of endothelial cells that perform different functions, with the inner wall being more resistant to drainage.

The uveoscleral pathway starts at the anterior ciliary body, passes through the ciliary muscle, and enters the space between the ciliary body and sclera. It then penetrates the sclera and diffuses into tissue behind the eye, where it is absorbed by blood vessels and the lymphatic system.

Tenon's Capsule, or bulbar fascia, is a sheath between the conjunctiva and sclera that surrounds the eyeball. It connects to the conjunctiva at the front and merges with the sclera near the limbus, enabling smooth eye movement and supporting extraocular muscle function.

The corneal epithelium, the outermost layer of the cornea, serves as both a protective barrier and a critical layer for refractive function. It is a stratified, non-keratinized tissue consisting of basal, wing, and superficial squamous cells. Renewal of the corneal epithelium is a dynamic process, driven by LESCs that divide, migrate, and differentiate to maintain the corneal surface, renewing every 7-10 days. This process involves a hierarchy of clonal keratinocytes, with true stem cells (holoclones) being the least abundant but most potent.

The conjunctiva is a thin, transparent mucous membrane divided into palpebral, bulbar, and fornix regions. Conjunctival epithelial stem cells (CESCs) are primarily found in the fornix and palpebral conjunctiva, where they give rise to both goblet and non-goblet epithelial cells, essential for maintaining the tear film and ocular surface health. These stem cells are typically quiescent, dividing slowly to preserve long-term regenerative capacity.

### Glaucoma Eye Drops

Glaucoma, a major cause of blindness, results from optic nerve damage due to high intraocular pressure (IOP). Lowering IOP is currently the only effective treatment. Aspects of the present invention describe the potential of combining ergothioneine and spermine with prostaglandin analogs or other uveoscleral modulators to create new glaucoma eye drops.

Ergothioneine accumulates in ocular tissues and reduces oxidative stress and inflammation, supporting trabecular meshwork (TM) function in glaucoma. Spermine, which is decreased in the aqueous humor of glaucoma patients, helps alleviate ER stress, supports protein folding, and provides anti-fibrotic and anti-inflammatory benefits for TM cells by addressing protein misfolding and ECM fibrosis.

Combining ergothioneine and spermine may enhance the TM-SC pathway and overcome limitations of traditional glaucoma treatments. Pairing this strategy with drugs that improve uveoscleral or conventional outflow (like PGA, Rho kinase inhibitors, nitric oxide donors, or adenosine receptor agonists) could offer multi-target intraocular pressure reduction and optic nerve protection for a more effective glaucoma solution.

### Glaucoma Pathophysiology: Aqueous Humor Dynamics and Molecular Mechanisms

IOP is regulated by the balance between aqueous humor production by the ciliary body and its drainage through the trabecular meshwork-Schlemm's canal (conventional) and uveoscleral (non-conventional) pathways. In primary open-angle glaucoma, impaired drainage raises intraocular pressure, damaging the optic nerve and causing permanent vision loss.

### A. Dysfunction of the Traditional (Trabecular Meshwork-Schlemm's Canal) Outflow Pathway

The trabecular meshwork regulates the drainage of aqueous humor from the anterior chamber of the eye, acting as a key determinant of intraocular pressure (IOP). In humans, the conventional outflow pathway—including the trabecular meshwork and Schlemm's canal—is the primary route for aqueous humor drainage, accounting for approximately 60% to 80% (in some sources, 70% to 90%) of total outflow. In primary open-angle glaucoma (POAG), elevated IOP is mainly due to increased resistance to aqueous humor drainage through the trabecular meshwork and/or Schlemm's canal. Most of the outflow resistance occurs in the juxtacanalicular zone of the trabecular meshwork and the endothelium of Schlemm's canal.

Trabecular meshwork cells possess contractile properties similar to smooth muscle cells, which influence outflow resistance. Alterations in the extracellular matrix (ECM), detected by integrins, are closely associated with trabecular meshwork cell contractility and outflow regulation. Glaucomatous trabecular meshwork tissue shows increased stiffness and changes in ECM composition, particularly an increase in fibrous ECM in the JCT region. Higher levels of transforming growth factor-beta 2 (TGF-B2) found in the aqueous humor of POAG patients promote enhanced ECM production and trabecular meshwork fibrosis, contributing to increased outflow resistance.

Dysfunction in the trabecular meshwork-Schlemm's canal pathway in glaucoma involves cellular mechanics, ECM integrity, and fibrotic processes. This indicates that therapies targeting only single mechanisms may not provide optimal efficacy, suggesting the need to explore multi-target combination therapies. Maintaining normal aqueous humor outflow resistance depends on both the contractile state of trabecular meshwork cells and ECM remodeling. Integrin signaling affects trabecular meshwork cell contractility, while the contractile state of these cells also influences ECM composition. Under pathological conditions, increased and stiffened ECM—driven by factors such as TGF-B2—can further modify cellular mechanics. This establishes a cycle where ECM stiffening leads to cellular alterations, resulting in additional pathological ECM deposition and greater outflow resistance. Therapeutic interventions should aim to disrupt this process. Since most outflow resistance exists within the TM outflow pathway upstream of SC, especially at the lining endothelium and underlying juxtacanalicular connective tissue, drugs designed to improve conventional outflow must demonstrate effectiveness at these specific sites. Drug design should therefore consider both biochemical pathways and pharmacokinetic features to maintain adequate concentrations and activity in these critical microanatomical regions.

B. Non-Traditional (Uveoscleral) Outflow Pathways and their Role in IOP Regulation

Aqueous humor also drains out of the eye via the uveoscleral (non-traditional) route, which involves the escape of aqueous humor through the ciliary muscle gap into the suprachoroidal space. This route accounts for 10% to 40% (in some estimates 10% to 30%) of total aqueous humor outflow, with a higher proportion in younger individuals but decreasing with age. Unlike trabecular meshwork outflow, uveoscleral outflow is largely unaffected by IOP within the normal physiological IOP range. Ciliary muscle contraction decreases uveoscleral fluid flow, while relaxation increases fluid flow.

Prostaglandin F2alpha (PGF2alpha) and its analogs (PGAs) are first-line drugs for glaucoma, which mainly increase uveoscleral outflow by binding to FP receptors in the ciliary muscle, leading to ECM remodeling and gap widening.

Uveoscleral outflow declines with age, presenting a specific therapeutic opportunity for elderly patients with glaucoma. The continued decrease in uveoscleral outflow with age suggests that this pathway is increasing impaired in the population most severely affected by glaucoma. Therefore, drugs that effectively enhance uveoscleral outflow could offer significant benefits to elderly patients, potentially restoring more youthful outflow dynamics and providing a potent mechanism for lowering IOP to complement or compensate for age-related declines in traditional pathways. While PGA is a well-established therapeutic agent, its mechanism of action (ECM remodeling) suggests that novel drugs that affect ciliary muscle ECM dynamics may offer new therapeutic avenues. Understanding the mechanism by which PGA increases uveoscleral outflow by remodeling the ciliary muscle's ECM provides a foundation for the development of novel compounds with similar ECM-remodeling effects but potentially different pharmacological properties or fewer side effects. For example, drug targeting ECM-degrading enzymes (such as matrix metalloproteinases (MMPs)) or ECM-synthetic pathways could be explored to achieve more refined or more effective uveoscleral outflow regulation.

### The Role of Ergothioneine in Eye Health and Glaucoma

### A. Ergothioneine as a Potent Antioxidant and Cytoprotectant

EGT is a thiohistidine betaine amino acid not synthesized by mammals or plants, and is mainly obtained from the diet. As a potent antioxidant, it neutralizes singlet oxygen, hydroxyl radicals, and peroxynitrite, and accumulates in tissues experiencing oxidative stress, including ocular areas. The OCTN1 transporter enables high cellular EGT concentrations at sites of injury or inflammation. EGT also prevents protein nitration, maintains lactoferrin's antimicrobial action, regulates antioxidant enzymes, and protects nitric oxide. In animal models, EGT reduces retinal neuron loss and shows neuroprotective effects.

EGT's antioxidant and anti-inflammatory actions make it a promising candidate for treating oxidative stress and inflammation in glaucoma, beyond simply lowering intraocular pressure (IOP). Its sulfhydryl group imparts broad cytoprotective, detoxifying, and anti-inflammatory benefits, inhibiting NF-kB activation and IL-8 release induced by oxidative stress.

OCTN1-driven accumulation allows EGT to selectively reach damaged ocular tissues, with elevated levels found in inflamed regions. This targeted mechanism may enhance efficacy against glaucoma-related damage in structures like the trabecular meshwork and retina while minimizing systemic side effects.

### B. Potential Role of Ergothioneine in Trabecular Meshwork Function and Outflow

Oxidative stress and endoplasmic reticulum (ER) stress interact to contribute to glaucomatous changes in the trabecular meshwork (TM), retinal ganglion cells, and optic nerve head. Trabecular meshwork cells are influenced by oxidative, mechanical, and phagocytic stress. EGT has been observed to inhibit oxidative stress-induced NF-kB activation and IL-8 release, indicating an anti-inflammatory response. Additionally, EGT offers protection to endothelial cells against oxidative stress, which is relevant for the health and function of TM cells. The extracellular matrix (ECM) of the TM undergoes regular turnover and remodeling, and the maintenance of intraocular pressure (IOP) homeostasis appears linked to ECM turnover.

EGT interacts with pathological mechanisms within the TM by reducing oxidative stress and inflammation, which may enhance its function. Individuals with glaucoma exhibit increased homocysteine concentrations in aqueous humor and plasma, contributing to oxidative stress in TM cells and reducing their proliferation. As an antioxidant, EGT counteracts this damage by inhibiting reactive oxygen species production and supporting the expression of antioxidant enzymes. By addressing oxidative stress in TM cells, EGT may assist in restoring normal cell functions, including phagocytic activity and responsiveness to mechanical cues, both important for homeostasis of aqueous humor outflow resistance.

The antioxidant properties of EGT may also influence ECM turnover, potentially limiting fibrotic changes. Altered remodeling and hardening of the ECM in TM play a role in increased outflow resistance in glaucoma. While direct effects of EGT on ECM remodeling are not established in current data, its antioxidant and anti-inflammatory actions may reduce cellular stress that leads to ECM damage and fibrosis. Oxidative stress can interfere with protein folding mechanisms, which depend on redox balance. By supporting intracellular redox equilibrium, EGT may aid TM cells in synthesizing and degrading ECM components appropriately, preventing atypical deposition and tissue stiffening. This indirect effect on ECM integrity is relevant for preserving the flexibility and permeability of the TM, facilitating aqueous humor outflow.

### The Role of Spermine in Eye Health and Glaucoma

### A. Spermine as a Polyamine and Chemical Chaperone

Spermine is a polyamine found in all life forms that supports DNA repair and acts as a chemical chaperone, enhancing protein folding and stability. It alleviates endoplasmic reticulum (ER) stress—a factor in myosin-related glaucoma and glucocorticoid-induced ocular hypertension—by stabilizing mutant proteins and promoting their secretion, helping rescue glaucomatous phenotypes. Spermine also protects nerve cells by reducing oxidative stress, inhibiting ASK1 activity, and suppressing monocyte-driven inflammation.

As an antioxidant and anti-inflammatory agent, spermine addresses key issues in glaucoma, notably protein misfolding and mitochondrial dysfunction. By assisting protein folding and clearing misfolded proteins, spermine may restore proteostasis, improve cell function, and delay TM cell death. Studies show that chemical chaperones like spermine and PBA can alleviate ER stress and improve outcomes in glaucoma models. Unlike conventional therapies that lower intraocular pressure, spermine targets cellular pathology directly, making it a promising treatment for glaucoma linked to protein folding defects.

### B. Direct Effects of Spermine on the TM and Aqueous Humor Outflow

Metabolomics analysis identified reduced spermine concentrations in the aqueous humor of patients with POAG. Spermine is associated with neuroprotection and cell membrane remodeling. TM cells display contractile properties and contribute to extracellular matrix (ECM) turnover. Transforming growth factor-beta (TGF-beta) signaling is known to decrease intracellular polyamine levels and facilitate epithelial-mesenchymal transition (EMT); application of exogenous spermine has been found to mitigate TGF-beta-induced EMT through reduction of fibronectin expression.

The observed spermine deficiency in the aqueous humor of glaucoma patients indicates that spermine supplementation may address a specific biochemical alteration. Research shows that spermine levels are significantly lower in the aqueous humor of individuals with POAG, correlating with changes in metabolites related to osmoprotection, neuroprotection, and cell membrane remodeling. This shortage may represent a disruption in polyamine metabolism in glaucoma pathology or reflect polyamine depletion under cellular stress. Exogenous spermine supplementation could potentially offset this deficit and support functions such as cellular homeostasis, protein folding, and regulation of the ECM. This approach seeks to correct a specific pathological change and may serve as a complement to treatments focused on lowering intraocular pressure (IOP).

Spermine's capacity to counteract TGF-beta-induced epithelial mesenchymal transition (EMT) and reduce fibronectin expression is considered relevant for improving TM outflow by limiting fibrosis and stiffness. Elevated TGF-beta2 concentrations have been reported in the aqueous humor of glaucoma patients, contributing to TM fibrosis and increased outflow resistance. Evidence suggests TGF-beta signaling leads to decreased polyamine levels and promotes EMT, while exogenous spermine can moderate these effects by downregulating ECM proteins such as fibronectin. Fibronectin accumulation within the TM is characteristic of glaucomatous eyes, and its reduction may help restore TM flexibility and permeability. By targeting TGF-beta-mediated fibrosis, spermine may influence TM structure and function, potentially enhancing aqueous humor outflow.

### Potential of Ergothioneine and Spermine to Synergistically Improve the Trabecular Meshwork-Schlemm's Canal Pathway

Oxidative stress and ER stress together contribute to glaucoma-related damage in the trabecular meshwork (TM), retinal ganglion cells, and optic nerve head. EGT acts as an antioxidant and cytoprotectant, reducing oxidative stress and inflammation, while spermine, a chemical chaperone, alleviates ER stress, enhances protein folding, and offers anti-inflammatory and antioxidant benefits. Spermine also prevents fibrosis by attenuating TGF-beta-induced EMT and lowering fibronectin levels. TM cell contractility, influenced by nitric oxide and endothelin-1, affects outflow resistance.

Combining EGT and spermine may have synergistic effects against glaucoma by targeting oxidative stress, ER stress, and inflammation, thus protecting TM cells and promoting cell viability. EGT defends against oxidative damage, while spermine supports protein homeostasis and reduces fibrosis, potentially restoring TM function and improving aqueous humor outflow more effectively than monotherapy. Both compounds show neuroprotective properties, supporting their potential as a combination therapy.

Additionally, pairing EGT, spermine, and prostaglandin analogs could further enhance total aqueous humor outflow via both conventional and non-conventional pathways.

### Uveoscleral Access Improvement: Current and NEW Methods

A. Prostaglandin Analogs (PGAs) and their Mechanisms

Prostaglandin analogs (PGAs) are commonly used as the initial treatment for glaucoma, reducing intraocular pressure (IOP) by increasing uveoscleral outflow through extracellular matrix (ECM) remodeling. Certain PGAs, such as latanoprost butanediol, also enhance outflow via nitric oxide-mediated relaxation of trabecular meshwork (TM) cells. PGAs are characterised by once-daily dosing, a favourable safety profile, and generally high compliance, although individual patient response may differ.

PGAs may lead to local side effects including conjunctival hyperemia, eyelash growth, and iris pigmentation, which have the potential to affect compliance and quality of life. These considerations have led to interest in alternative or combination therapies. Current research in glaucoma management is focused on developing drugs with novel mechanisms of action or reduced side effect profiles.

### B. Novel Non-Prostaglandin Uveoscleral Outflow Enhancers

In addition to PGA, some newer drugs also enhance aqueous humor outflow or provide neuroprotection through different mechanisms.

Rho kinase (ROCK) inhibitors. Rho kinase inhibitors, including Ripasudil and Netarsudil, reduce intraocular pressure by relaxing trabecular meshwork cells via Rho/ROCK pathway inhibition, which enhances aqueous humor outflow. Netarsudil also lowers episcleral venous pressure, improving distal drainage routes. Unlike prostaglandin analogs that boost uveoscleral outflow, these drugs directly target conventional pathways, providing a complementary approach to glaucoma management.

Nitric oxide (NO) donors. Nitric oxide (NO) is a natural signaling molecule that regulates intraocular pressure (IOP), improves ocular blood flow, and protects nerves. NO donors like latanoprost butanediol ester (LBN) reduce IOP by changing cell volume and increasing uveoscleral outflow. They work through the SGC-cGMP-PKG pathway, leading to TM cell relaxation and lower outflow resistance, and also support retinal ganglion cell health. The market success of LBN highlights the potential of NO donors as glaucoma treatments, combining dual outflow benefits with neuroprotection.

Adenosine receptor (AR) agonists. Adenosine agonists lower intraocular pressure (IOP) by reducing aqueous humor production initially and later increasing outflow, mainly via activation of the A1 adenosine receptor. This process involves matrix metalloproteinases (MMPs), which facilitate outflow. Additionally, these agonists decrease vascular resistance, improve retinal and optic nerve head blood flow, and support retinal function recovery after ischemia. Their combined pressure-lowering and neuroprotective effects offer a non-PGA treatment option for glaucoma by regulating IOP and enhancing ocular health.

Endothelin (ET-1) receptor antagonists. Endothelin-1 (ET-1) receptor antagonists represent a class of agents targeting a potent vasoconstrictor peptide that is elevated in the aqueous humor of patients with primary open-angle glaucoma (POAG), resulting in increased intraocular pressure (IOP) and reduced ocular blood flow. Inhibition of ET-1 signaling can lower IOP and offer neuroprotective benefits.

Unlike conventional treatments focused solely on IOP reduction, endothelin receptor antagonists address additional pathophysiological mechanisms by modulating vascular tone, enhancing ocular perfusion, and conferring neuroprotection. Elevated ET-1 concentrations observed in glaucoma correlate with its potent vasoconstrictive effects via both ETA and ETB receptors. ET-1 influences IOP through trabecular meshwork contractility and also compromises ocular blood flow, contributing to pathological alterations in the retina and optic nerve head that may result in retinal ganglion cell degeneration. Thus, dual endothelin antagonists, which inhibit ET-1 signaling, have the potential to mitigate vasoconstriction, improve ocular blood supply, and directly protect retinal ganglion cells. This therapeutic approach may be especially advantageous for glaucoma patients experiencing progressive optic nerve damage despite normal IOP, or those requiring comprehensive multi-targeted interventions.

### Strategic Recommendations for the Development of New Glaucoma Eye Drops

A. EGT Combining with Spermine Enhances TM-SC Pathway Outflow

Combining EGT and spermine may enhance TM-SC pathway function by improving aqueous humor outflow through synergistic effects. EGT, an oxidant and cytoprotectant, protects TM cells from oxidative stress and inflammation, preserving cellular integrity and filtration efficiency. Spermine, deficient in glaucoma patients, reduces ER stress, supports protein folding, and counters fibrosis, thereby lowering TM stiffness. Together, EGT addresses external cellular stress while spermine manages internal processes, offering a comprehensive approach to TM health. Although direct effects on uveoscleral outflow are unclear, the improved ocular microenvironment could indirectly benefit this pathway. This combination with prostaglandin analogs may lead to significant advancements in glaucoma treatments.

B. Combination with Prostaglandin Analogs or Other Uveoscleral Enhancers

To improve glaucoma eye drops, combining EGT and spermine with current or novel IOP-lowering drugs may offer multi-target, multi-mechanism treatment effects.

Option 1: EGT+Spermine+PGA (e.g., latanoprost Butanediol). Latanoprost butanediol ester (LBN) is a distinct PGA that boosts uveoscleral outflow via FP receptor activation and enhances conventional outflow through TM cells by donating nitric oxide. Combining EGT and spermine with LBN improves TM cell health, protein balance, and anti-fibrosis, further increasing LBN's efficacy. They also offer neuroprotection, essential for glaucoma patients' long-term vision. This approach delivers multimodal therapy: lowering IOP, promoting neuroprotection, and providing anti-inflammatory and anti-fibrotic benefits.

Option 2: EGT+Spermine+Rho kinase inhibitor (e.g., Ripasudil, Netarsudil). Rho kinase inhibitors lower outflow resistance by regulating the TM cytoskeleton and EMC. Combining EGT and spermine with Rho kinase inhibitors produces a synergistic effect: EGT protects TM cells from stress, while spermine improves TM structure and function. This approach effectively targets pathological TM changes and enhances conventional outflow, with potential neuroprotective benefits.

Option 3: EGT+Spermine+Nitric oxide donor (pure compound, not PGA). Pure nitric oxide donors can directly relax TM cells, increase aqueous humor outflow, and have neuroprotective effects. Combining EGT and spermine with pure nitric oxide donors can further enhance the effects of improved aqueous humor outflow. By improving the overall health and protein homeostasis of TM cells, EGT and spermine provide a healthier cellular environment for the effects of nitric oxide. This combination is expected to achieve robust enhancement of aqueous humor outflow while providing comprehensive cellular and neuroprotective effects.

Option 4: EGT+Spermine+Adenosine Receptor Agonist. Adenosine receptor agonists lower IOP through reduced aqueous humor production initially and increased outflow later, while also offering neuroprotection. Adding EGT and spermine supports TM cell health and protein balance, potentially boosting aqueous humor outflow. Together, all three ingredients offer broad protection for retinal ganglion cells.

### C. Formulation and Delivery Considerations

Topical eye drops remain the primary method for administering glaucoma therapy; however, their effectiveness is hindered by factors such as limited bioavailability, insufficient drug penetration, and rapid tear clearance. Achieving efficient delivery of EGT, spermine, and their combination to relevant ocular tissues (trabecular meshwork, ciliary body, etc.) necessitates rigorous formulation optimization.

Nanoparticle-based formulations offer notable advantages, including enhanced dispersibility in aqueous media and improved transmembrane penetration across lipophilic biological barriers, thereby increasing bioavailability and prolonging drug residence time. Solid drug nanoparticles (SDNs), characterized by high drug loading and carrier-free attributes, are particularly suitable for delivering hydrophobic agents. EGT demonstrates favourable ocular permeability, with substantial accumulation observed in the cornea and aqueous humor. However, optimal nanoformulation strategies for spermine and its combination with EGT require further investigation to ensure effective targeting of key anatomical sites, such as the trabecular meshwork and ciliary body.

While fixed-combination eye drops provide convenience for patients, they may restrict flexibility in dosing and timing of individual constituents. The development of EGT and spermine combination eye drops should carefully balance patient convenience with individualized therapeutic needs. Future research ought to prioritize innovative formulations that enable targeted delivery, sustained release, and maximized bioavailability, thus enhancing the therapeutic efficacy of these compounds.

Subsequent publications will concentrate on the detailed formulation approaches involving EGT and spermine alongside various prostaglandin analogs and uveoscleral outflow enhancers, with particular emphasis on elucidating mechanisms underlying synergistic effects.

### Conclusions

Aspects of the present invention explore the potential of EGT and spermine for new glaucoma eye drops, focusing on their combined ability to enhance TM-SC pathway function. EGT acts as an antioxidant and cytoprotectant, countering oxidative stress and inflammation, while spermine addresses protein misfolding and ECM fibrosis. Together, they aim to restore TM health and improve aqueous humor outflow.

Combining EGT and spermine with agents that boost uveoscleral outflow (like PGA, Rho kinase inhibitors, nitric oxide donors, or adenosine receptor agonists) offers a promising multi-target approach, improving outflow and providing neuroprotection beyond IOP reduction.

Treatment of Glaucoma with Chemical Chaperones

Chemical chaperones, including small molecules that support protein folding and stability, are promising for correcting cellular dysfunctions such as ER stress and protein imbalance.

Studies show 4-phenylbutyric acid (4-PBA) effectively reduces IOP via the trabecular meshwork-Schlemm's canal (TM-SC) pathway. In contrast, spermine mainly provides neuroprotection to retinal ganglion cells without affecting IOP, indicating it cannot substitute for 4-PBA in TM-SC pathway improvement.

The feasibility of chemical chaperone-based PGA replacements lies not in mimicking PGA's receptor-mediated extracellular matrix (ECM) remodeling, but rather in addressing cellular pathologies upstream of the trabecular meshwork that contribute to aqueous humor outflow resistance, such as protein misfolding and endoplasmic reticulum stress. By restoring the health and function of trabecular meshwork cells, chemical chaperones offer a mechanistically distinct yet therapeutically similar approach to lowering intraocular pressure. Furthermore, other natural compounds with antioxidant and anti-inflammatory properties, often closely linked to protein homeostasis, offer further avenues for multi-targeted glaucoma treatment.

### Role of Protein Misfolding and Endoplasmic Reticulum (ER) Stress in the Pathogenesis of Glaucoma

Protein misfolding and mitochondrial dysfunction have been identified as contributing factors in open-angle glaucoma. The endoplasmic reticulum (ER) serves as a protein quality control system. An accumulation of misfolded or unfolded proteins disrupts ER homeostasis and leads to ER stress. This stress initiates the unfolded protein response (UPR), which aims to restore protein homeostasis; persistent stress, however, may result in apoptosis.

Mutations in myosin (MYOC) are recognized as the most prevalent single cause of POAG. Mutant myosin accumulates in the ER, activating ER stress and causing trabecular meshwork (TM) cell death. Both oxidative stress and ER stress together contribute to damage in the TM, retinal ganglion cells, and the optic nerve head associated with glaucoma. The connection between protein misfolding, ER stress, and TM cell death highlights a central pathological pathway in glaucoma, especially in cases related to myosin mutations, and supports the rationale for interventions with chemical chaperones, which target protein folding and ER homeostasis.

### Current First-Line Drug Treatment: Prostaglandin Analogs (PGAs)

Prostaglandin analogs (PGAs) are widely recognized as the first-line therapeutic agents for glaucoma due to their proven efficacy in lowering intraocular pressure (IOP), convenient once-daily administration, and generally favorable side effect profile. These medications are 20-carbon fatty acid derivatives, biosynthesized from arachidonic acid through the cyclooxygenase (COX) pathway.

PGAs decrease IOP primarily by enhancing aqueous humor outflow, predominantly via the uveoscleral pathway and, to a lesser extent, through the conventional trabecular meshwork-Schlemm's canal (TM-SC) route. Their pharmacological action involves binding to prostaglandin FP receptors located in the ciliary muscle, which results in extracellular matrix (ECM) remodeling and relaxation of the ciliary muscle. This process expands the interstitial space, thereby facilitating increased aqueous humor outflow. Additionally, PGAs promote the synthesis of matrix metalloproteinases (MMPs), leading to ECM degradation and reduced outflow resistance.

The mechanisms of PGAs—comprising receptor-mediated signaling, ECM remodeling, and smooth muscle relaxation—differ fundamentally from the protein folding and endoplasmic reticulum (ER) stress pathways targeted by chemical chaperones. Consequently, PGAs do not directly address protein misfolding or ER stress. Chemical chaperones, therefore, represent a novel class of IOP-lowering agents that target cellular proteostasis rather than receptor signaling pathways. This distinction highlights opportunities for combination therapies and addresses the needs of patients who may be unresponsive to PGAs.

### Chemical Chaperone Theory: Principles and Relevance to Glaucoma

### A. Core Concepts of Chemical Chaperones: Mechanism of Action and Classification

Chemical chaperones (CCs) constitute a group of small molecules that promote protein folding and/or stability. Unlike molecular chaperones—large protein complexes such as HSP70 and HSP90—chemical chaperones function through distinct mechanisms affecting protein stability and polypeptide organization.

Classification and Mechanism. Osmolytes are small polar molecules (e.g., glycerol, trehalose, trimethylamine N-oxide [TMAO], glycine) that act indirectly by modifying protein-solvent interactions. Their presence enhances protein hydration, favouring a compact, folded conformation. For instance, TMAO expedites refolding by constraining the flexibility of intermediate protein states. Hydrophobic Compounds include agents with varying hydrophobicity and solubility in aqueous environments (such as 4-phenylbutyric acid [PBA], lysophosphatidic acid, lipids, and detergents), these compounds bind to solvent-exposed hydrophobic regions of unfolded or misfolded proteins, thereby preventing aggregation. Pharmacological chaperones encompass protein ligands, cofactors, competitive inhibitors, and other small molecules that selectively bind to specific proteins. Their stabilizing effects arise from favorable binding interactions, which reduce the conformational flexibility of targeted regions within the polypeptide chain.

Chemical chaperones inhibit intracellular aggregation of misfolded proteins and restore trafficking, non-specifically stabilizing native conformations and facilitating escape from endoplasmic reticulum (ER) quality control systems. Their classification-osmolytes, hydrophobic compounds, and pharmacological chaperones-highlights mechanistic diversity. Thus, the chemical chaperone approach represents a spectrum rather than a singular model, with certain classes potentially more effective for specific misfolding events or cellular environments. For example, the hydrophobic nature of 4-PBA indicates its affinity for exposed hydrophobic regions implicated in protein aggregation. An in-depth understanding of these mechanisms is essential for the discovery of new chemical chaperones. The chemical structure of a compound may predispose it towards a particular chaperone activity, informing the rational design of targeted therapeutics for protein misfolding diseases, such as myosin-related glaucoma.

### B. Unfolded Protein Response (UPR) and ER Stress in Ocular Diseases

When the endoplasmic reticulum (ER) reaches its limit for proper protein folding, misfolded and unfolded proteins build up, resulting in ER stress. The unfolded protein response (UPR) acts to restore protein balance by reducing protein translation, enhancing protein folding, and increasing protein degradation. Prolonged ER stress can lead to cell apoptosis. ER stress plays a significant role in the development of several ocular diseases, including glaucoma. Chronic ER stress has been identified in human glaucomatous trabecular meshwork (TM) tissue and cells, where sustained ER stress leads to TM cell apoptosis and contributes to glaucoma progression.

The UPR exhibits both adaptive and pro-apoptotic functions depending on whether stress is alleviated, presenting an opportunity for chemical chaperone therapy. The therapeutic objective involves modulating the UPR to favor adaptive survival pathways over apoptotic ones, thereby aiming to restore TM function and prevent cell death. Drug development focuses on refining the cellular response rather than simply alleviating ER stress. Effective chemical chaperones may support the pro-survival aspect of the UPR or inhibit signaling involved in apoptosis, helping maintain TM cell viability and function, which is important for long-term intraocular pressure control.

### C. 4-PBA as a Pioneering Chemical Chaperone for Glaucoma Treatment

4-PBA, an FDA-approved drug with chaperone properties, helps reduce ER stress by aiding protein folding. In a POAG mouse model (Tg-MYOC-Y437H), oral 4-PBA reduces ER stress caused by mutant myosin buildup, preserves TM cells, and normalizes IOP, preventing retinal ganglion cell degeneration. These results demonstrate that targeting protein homeostasis can effectively manage IOP by improving the TM-SC outflow pathway, supporting chemical chaperones as a promising treatment for glaucoma through a novel mechanism distinct from current therapies.

Re-Evaluation of Spermine as a Glaucoma Chemical Chaperone: Comparison with 4-PBA

### A. Polyamines (Spermine and Spermidine): Biological Effects and Properties

Spermine is classified as a tetraamine and spermidine as a triamine; both are low molecular weight linear polyamines found universally across life forms. Spermidine functions as a precursor to spermine. Polyamines play diverse metabolic roles, such as regulating ion channels, facilitating DNA repair, and modulating cell growth, proliferation, and apoptosis. Notably, spermidine has been identified as a potential agent for lifespan extension in mammals, primarily through the induction of autophagy, and secondarily by attenuating inflammation and influencing lipid metabolism.

Spermine exhibits extensive and foundational biological activity that reaches beyond its involvement in protein folding. The broad scope of its effects implies possible pleiotropic roles in disease processes, but also indicates that its “chemical chaperone” activity may be integrated within a more intricate framework of cellular regulation. This complexity can complicate efforts to delineate specific mechanisms relevant to glaucoma therapy. Although autophagy is integral to protein homeostasis, it encompasses a wider spectrum of cellular clearance processes. Therefore, if spermine operates as a chemical chaperone, it likely does so through indirect or systemic mechanisms that enhance overall protein homeostasis, such as promoting autophagy or exerting antioxidant effects, rather than acting as a targeted protein-binding chaperone like 4-PBA. Recognizing this complexity is essential when considering spermine's therapeutic potential as a replacement strategy.

### B. Evidence for the Regulatory Effects of Spermine on ER Stress and Oxidative Stress

Spermine acts as an antioxidant and Ca2+ chelator, helping prevent cell apoptosis by protecting against oxidative stress-induced Ca2+ overload. It lowers oxidative markers and ER stress-related proteins in cell models. In glaucoma, oxidative and ER stress collectively damage TM, retinal ganglion cells, and the optic nerve head, with ER stress activating the unfolded protein response (UPR). Spermine reduces 4-HNE levels in EAAC1 KO mice retinal ganglion cells.

While spermine decreases both ER stress proteins and oxidative stress, its main effects are antioxidant activity, Ca2+ buffering, and autophagy, indirectly regulating protein homeostasis. This broad cellular stress relief is different from 4-PBA's direct action as a hydrophobic chaperone.

### C. Distinguishing the Mechanisms of Action: The Neuroprotective Effects of Spermine and the IOP-Improving Effects of 4-PBA Via the TM-SC Pathway

Effect of 4-PBA: 4-PBA functions as a chemical chaperone, mitigating glaucomatous phenotypes by alleviating endoplasmic reticulum (ER) stress in the trabecular meshwork (TM). This action normalizes intraocular pressure (IOP) and prevents retinal ganglion cell (RGC) loss, thereby enhancing TM cell health and aqueous humor outflow.

Effects of spermine: Oral administration of spermine has been shown to counteract glaucomatous retinal degeneration in EAAC1KO mice without altering IOP levels. The neuroprotective effects of spermine on retinal ganglion cells are primarily attributed to its antioxidant properties, reduction of oxidative stress markers, and inhibition of ASK1 activity. Notably, spermidine and spermine predominantly accumulate within glial cells of the retina rather than neurons.

Contradiction with the hypothesis: Current evidence does not support the hypothesis that spermine can substitute for 4-PBA in improving the TM-SC pathway. While both compounds address cellular stress, 4-PBA exerts a direct impact on IOP via the TM, whereas spermine's core documented benefit is neuroprotection, independent of IOP regulation.

This distinction is pivotal in addressing whether spermine can replace 4-PBA, as the mechanisms and outcomes differ markedly—4-PBA reduces IOP through regulation of TM-ER stress, while spermine offers neuroprotection without influencing IOP. Although both agents may be regarded as “chemical chaperones” or “cellular stress regulators,” their roles in glaucoma pathology are distinct.

The therapeutic applications and molecular targets of these compounds are not interchangeable; spermine does not directly substitute for 4-PBA in modulating the TM-SC pathway to lower IOP. These findings underscore that the “chemical chaperone theory” encompasses a spectrum of therapeutic potentials within a single disease context, necessitating precise characterization of each compound's primary targets and clinical benefits. Moreover, this highlights prospects for combination therapies directed at both IOP control and neuroprotection.

TABLE 2
Mechanisms of Action for 4-PBA, Spermine, and PGAs in Glaucoma

| | Drug | | |
|---|---|---|---|
| Characteristics | 4-PBA | Spermine | PGAs |
| Class | Hydrophobic | Polyamines | Fatty Acid Derivatives |
| Mechanism | Reduces ER stress | Helps protein | Ca²⁺ chelation |
| | | folding | |
| Target tissue | TM/SC | RGCs/Glial cells | Ciliary muscle, TM |
| IOP Effect | Normalizes IOP | No direct IOP | Lowers IOP |
| | | effect | |
| AH outflow | Improves TM-SC | No evidence | Increases outflow |
| | flow | | |
| Neuroprotection | Prevents RGC loss | Protects | Indirect protection |
| | | RGCs/retina | |
| Relevance | Improves TM-SC | Not alternative to | Different approach |
| | path | 4-PBA | |

### Strategies for Discovering Novel Chemical Partners as Alternatives to PGAs

### A. Detailed Mechanism of Action of PGAs in Lowering IOP

PGAs like latanoprost, travoprost, and bimatoprost are PGF2 prodrugs that lower intraocular pressure by activating the prostaglandin FP receptor in the ciliary muscle. This triggers ECM remodeling and increases aqueous humor outflow, mainly through the uveoscleral pathway, and possibly relaxes the TM to boost SC permeability.

PGAs lower IOP via targeted receptor-mediated signaling and ECM changes but do not directly address ER stress or TM cell dysfunction involved in glaucoma. Chemical chaperones may complement or provide alternatives by targeting these underlying cellular problems, potentially offering more effective treatments for patients unresponsive to PGAs.

### B. Reasons for Seeking Alternative Glaucoma Treatments

While prostaglandin analogues (PGAs) are generally effective, a subset of patients are classified as non-responders, with efficacy varying considerably due to genetic factors. Additionally, PGAs may produce local side effects such as hyperemia, eyelash growth, and changes in iris pigmentation. Given that glaucoma is a multifactorial condition, current therapeutic strategies predominantly target intraocular pressure (IOP) reduction, leaving other pathogenic mechanisms-such as protein misfolding, oxidative stress, and neuroinflammation-unaddressed. There remains a significant clinical need for disease-modifying agents capable of targeting these additional pathways beyond merely regulating IOP.

The pursuit of alternative therapies for glaucoma extends beyond the development of new IOP-lowering medications; it encompasses the creation of treatments that address the complex pathophysiology of the disease. This includes factors such as protein misfolding, endoplasmic reticulum (ER) stress, oxidative stress, and neuroinflammation. Chemical chaperones present a promising approach by enhancing cellular proteostasis and targeting these interconnected pathological processes, potentially delivering disease-modifying benefits not observed with PGAs. Glaucoma's characterization as a multifactorial disease highlights the limitations of existing IOP-centric treatments and underscores the necessity for innovative therapies, such as chemical chaperones, which may offer more comprehensive disease management and improved patient outcomes by addressing multiple pathogenic mechanisms.

### C. Appling the Chemical Chaperone Principle to Target the Pathogenesis of Glaucoma Misfolded Proteins and ER Stress in TM Cells Increase Outflow Resistance and

cause cell death in glaucoma. Chemical chaperones can stabilize mutant proteins, promote proper folding and transport, and reduce toxicity. By restoring normal protein handling and reducing ER stress, they improve TM cell function and outflow, lowering IOP. This method addresses the underlying process of tissue stiffness and related signaling.

Rather than replacing lost function, chemical chaperones offer a new approach by directly targeting protein misfolding and ER stress at the root of glaucoma pathology. This may provide more lasting effects on disease progression than treatments like PGAs, which address downstream symptoms. By modifying disease mechanisms, chemical chaperones could maintain TM cell health and slow or prevent increased outflow resistance over time.

### D. Identification and Evaluation of Candidate Compounds: Beyond Traditional PGA Structures

“Proanalogs” refer to compounds that share similar chemical structures or pharmacological targets with established medications, or to prodrugs. Resoleic acid (RA), a naturally occurring non-prostaglandin analog, demonstrates dual agonist activity at both FP and EP3 receptors and exhibits intrahepatic pressure-lowering effects comparable to travoprost. Although RA is not classified as a chemical chaperone, it serves as an example of a non-PGA compound acting upon similar receptor targets.

Polyphenols: Ophthalmic formulations containing high concentrations of polyphenols have the potential to protect trabecular meshwork (TM) cells from oxidative stress. Due to the synergistic relationship between oxidative and endoplasmic reticulum (ER) stress in glaucoma, polyphenols—such as anthocyanins, Ginkgo biloba extract, EGCG, and resveratrol—may indirectly enhance protein homeostasis and support TM cell health. Notably, resveratrol mitigates chronic oxidative stress-induced expression of glaucoma markers and provides anti-apoptotic benefits in TM cells.

Ginkgo biloba: This botanical contains flavonoids with free radical scavenging properties, which can enhance retinal ganglion cell survival following oxidative insult, improve visual function in patients with normal-tension glaucoma (NTG), and increase ocular blood flow. Ginkgo biloba primarily confers neuroprotective and antioxidant effects, with its influence on cellular stress potentially offering indirect support for TM health.

Melatonin: As a novel neuroprotectant for glaucoma, melatonin preserves retinal ganglion cells via mitochondrial stabilization, protection against glutamate-induced neurotoxicity, and antioxidant pathways. Its mechanism highlights the importance of neuroprotection independent of intraocular pressure (IOP) modulation.

Other Natural Compounds: Agents such as baicalin, forskolin, ginsenosides, and hesperidin have demonstrated IOP-reducing effects, while curcumin, caffeine, coenzyme Q10, and vitamins B3/D have exhibited neuroprotective properties.

Xyloglucan: This polysaccharide, utilized in ophthalmic drug delivery systems, enhances bioavailability and sustained release, thereby improving drug efficacy without exerting direct therapeutic effects itself.

The investigation into “other ingredients” underscores a paradigm shift in glaucoma management towards holistic, multi-target strategies. Compounds including polyphenols and Ginkgo biloba offer therapeutic benefit through antioxidant and anti-inflammatory mechanisms intimately linked to ER stress and protein misfolding. This evidence suggests an expansion of the “chemical chaperone theory” beyond conventional protein folding interventions to encompass broader aspects of cellular proteostasis and redox balance, which contribute collectively to IOP regulation and neuroprotection. Many natural compounds exhibit antioxidant, anti-inflammatory, and anti-apoptotic activities, reinforcing the interdependence of ER and oxidative stress in glaucoma pathophysiology. Since protein folding depends on redox homeostasis, agents that attenuate oxidative stress—such as polyphenols in TM cells—can support proteostasis even without directly interacting with misfolded proteins. In sum, this perspective broadens the chemical chaperone concept to embrace comprehensive cellular resilience, enabling the identification of diverse compounds that promote TM and retinal ganglion cell health via multiple mechanisms, and thus foster multi-targeted approaches to glaucoma therapy.

Emerging Non-Prostaglandin Analogue Glaucoma Therapies with Potential Proteostasis Regulation

Polyphenols exhibit antioxidant, anti-inflammatory, and anti-apoptotic properties. These compounds help protect trabecular meshwork cells from oxidative stress and are considered neuroprotective. They may lower intraocular pressure (IOP) and can indirectly support protein homeostasis by mitigating oxidative stress, a factor in endoplasmic reticulum stress.

Ginkgo biloba functions as an antioxidant and anti-apoptotic agent and can improve ocular blood flow. Its primary benefits include protection of retinal ganglion cells and improvement of visual function. It has the potential to lower IOP and directly supports protein homeostasis by decreasing oxidative stress and enhancing cellular health.

Melatonin contributes to mitochondrial protection, acts as an antioxidant, and regulates neurotransmitters. Its main benefit is neuroprotection for retinal ganglion cells, though it does not have a direct impact on IOP. It addresses cellular stresses related to oxidation and mitochondria, which are relevant to protein homeostasis.

Baicalin, forskolin, ginsenosides, hesperidin demonstrate multiple mechanisms such as anti-inflammatory and antioxidant effects, and are associated with lowering IOP. Their influence on cellular stress pathways may be indirect.

Xyloglucan, a polysaccharide, enhances drug bioavailability and enables sustained release. It facilitates delivery of IOP-lowering medications, leading to an indirect reduction in IOP. While not a therapeutic agent itself, it serves as a delivery system that may promote the effectiveness of other treatments.

### Challenges and Future Directions of Glaucoma Therapy Based on Chemical Chaperones

### A. Specificity, Delivery, and Pharmacokinetics of Chemical Chaperones

Chemical chaperones act broadly, which can help with diverse protein misfolding but may cause depletion side effects. Pharmacological chaperones are more selective. Achieving sustained drug levels in the TM and retinal ganglion cells is important; topical delivery has poor permeability, but new formulations like xyloglucan gels improve retention and bioavailability. Understanding pharmacokinetics, such as plasma protein binding, is vital for drug efficacy.

### B. Combination Therapy and Multi-Target Approaches

Glaucoma is characterized by a multifactorial pathogenesis, involving several distinct biological processes. In the context of long-term management, therapies targeting multiple pathways may yield greater efficacy. The interrelationship between oxidative stress and endoplasmic reticulum (ER) stress indicates potential advantages for pharmacological approaches that address both mechanisms. A combination of chemical chaperones—targeting ER stress and protein misfolding—with neuroprotective agents such as spermine or melatonin, as well as traditional post-procedural glucocorticoids (PGAs), may offer a more comprehensive treatment strategy.

Given glaucoma's complexity and the diverse mechanisms of action exhibited by various drug classes (for example, PGAs facilitate aqueous humor outflow, chemical chaperones maintain protein homeostasis, and polyamines/melatonin provide neuroprotection), future therapeutic strategies are likely to emphasize combination regimens. Such approaches would move beyond mere “substitution” towards synergistic interventions, permitting each agent to target interconnected pathological pathways. Glaucoma encompasses a range of pathogenic factors-including elevated intraocular pressure (IOP), ER stress, oxidative damage, and neurodegeneration- and it is improbable that any single medication could adequately address all aspects of the disease. Therefore, future directions in glaucoma therapy, particularly those incorporating chemical chaperones, will probably focus on combining agents with complementary mechanisms of action. Examples include coupling a chemical chaperone to promote trabecular meshwork health with a neuroprotectant aimed at retinal ganglion cell preservation, or integrating a chemical chaperone with a PGA to optimize IOP reduction and cellular elasticity, thereby establishing a more sophisticated and potentially efficacious therapeutic paradigm.

### Conclusion

The chemical chaperone theory suggests a mechanistically distinct approach for glaucoma treatment, primarily by addressing protein misfolding and ER stress in the trabecular meshwork (TM), which may lead to reduced intraocular pressure (IOP) and improved aqueous humor outflow.

Research indicates that 4-PBA can lower IOP by improving the TM-Schlemm's canal pathway, while polyamines such as spermine are mainly associated with neuroprotective effects on retinal ganglion cells and do not directly affect IOP. This distinction clarifies initial hypotheses and demonstrates the various therapeutic possibilities within the framework of chemical chaperones.

Future research into alternatives to prostaglandin analogs may benefit from investigating compounds that promote cellular protein homeostasis and reduce TM cell stress, instead of focusing solely on replicating receptor agonist mechanisms. Natural compounds with antioxidant, anti-inflammatory effects and ER stress modulation could be considered for multi-targeted intervention.

Subsequent studies should aim to address translational challenges such as specificity, ocular delivery, and pharmacokinetics, while assessing the potential of combination therapies to manage the complex and multifactorial aspects of glaucoma. This approach may contribute to the development of new therapeutic strategies and an improved understanding of cellular pathology in glaucoma management.

### EXAMPLES

The following illustrative examples are representative of embodiments of the compositions, systems, and methods described herein that have been reduced practice, and are not meant to be limiting in any way.

### Example 1—Formulations, where EGT is an API

The ophthalmic formulation comprised: 0.5% (w/v) EGT as an active pharmaceutical ingredient (API) with: 0.05% (w/v), 0.01% (w/v), or 0% (w/v) of sodium hyaluronate; almost negligible amounts of taurine (0.01% (w/v), 0.03% (w/v), or 0.05% (w/v) of taurine); 0.5-0.9% (w/v) of sodium chloride; and the remainder is the pharmaceutically acceptable aqueous delivery vehicle, such that the overall water content of the formulation is 98-99.5% (w/v). In all the cases, sodium ions, from sodium hyaluronate and sodium chloride, were used to adjust the osmotic pressure of eye drops to the regular level. In the formulations that comprised 0.5% (w/v) EGT exhibited: high ROS scavenging efficiency; high potency; and no detectable irritation in the ocular tissue of the subjects. There were 88 subjects with dry eye, which manifested as one or more of the following: eye dryness, eye itching, eye redness, foreign body sensation, photophobia/wind sensitivity with tearing, sticky secretions, blurred vision, and painful sensation. The subjects were prompted to indicate the level of improvement of their symptoms related to dry eyes after consuming the formulation comprising EGT and whether the formulation comprising EGT provided a better experience than other eye drops or eye wash products. Of the 88 subjects, 20 subjects reported very obvious improvement in symptoms after using the formulation; 50 subjects reported obvious improvement in symptoms after using the formulation; 12 subjects reported some improvements in symptoms after using the formulation; 4 subjects reported not obvious improvements in symptoms after using the formulation; and 2 subjects reported there were little or no improvements in symptoms after using the formulation. Of the 88 subjects, 24 reported that the formulation was much better than prior used eye products; 51 reported that the formulation was better than prior used eye products; 13 reported that the formulation was the same as prior used eye products; and 0 reported that the formulation was not as good as prior used eye products.

### Example 2—Trial of L-EGT Eye Wash for Improving Ocular Discomfort

Two eye washes (or ophthalmic formulations) were prepared and investigated to: (1) study the effectiveness of EGT for improving ocular discomfort compared with the self-control group; and (2) observe the safety of EGT during the clinical trial. Eye Wash 1 was 5.0 mL (water, 0.5% EGT, sodium hyaluronate, taurine, and sodium chloride); and wash 2 was 0.5 mL (water, 0.5% EGT, sodium hyaluronate, taurine, and sodium chloride). Forty subjects completed the trial, where 20 subjects used wash 1 and 20 subjects used wash 2. In the safety data set of this trial, a total of 0 subjects experienced 0 adverse events, with an incidence rate of adverse events of 0%. No subject had adverse reactions or serious adverse events. The research methodology was: a single-center, randomized, double-blind, parallel, placebo-controlled clinical.

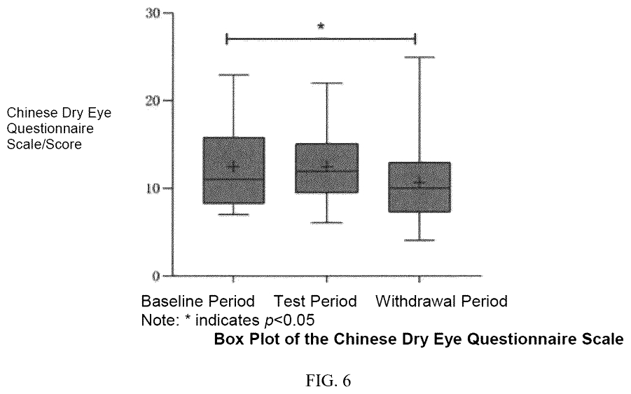

The mean value of the questionnaire was 12.50, and the mean value at the end-of-study period was 10.65, which decreased by 14.80%, and there was significant difference (P=0.0353), as depicted in FIG. 6. After the subjects were administered with EGT Eye Wash, the mean score of the Questionnaire Scale in the post-treatment period was significantly lower than that in the baseline period (P=0.0353).

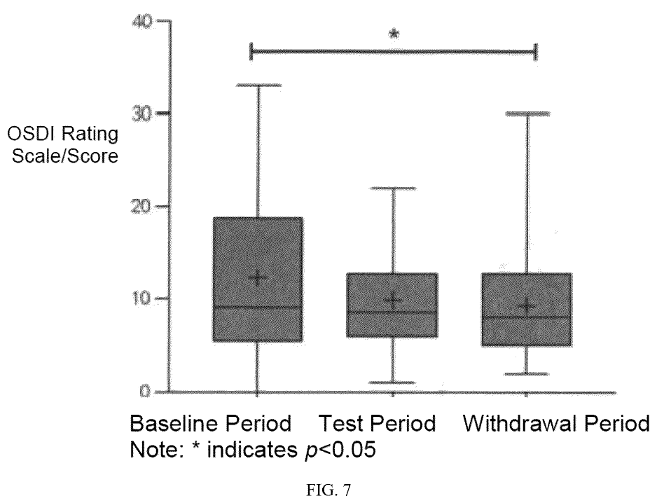

The mean value of the Ocular Surface Disease Index (OSDI) score of 40 subjects at the baseline period was 12.25, the mean value at the withdrawal period was 9.23, which decreased by 24.69%, and there was a significant difference (P=0.0309), as depicted in FIG. 7. After the subjects were administered with EGT Eye Wash, the mean score of the Ocular Surface Disease Index (OSDI) in the post-treatment period was significantly lower than that in the baseline period (P=0.0309).

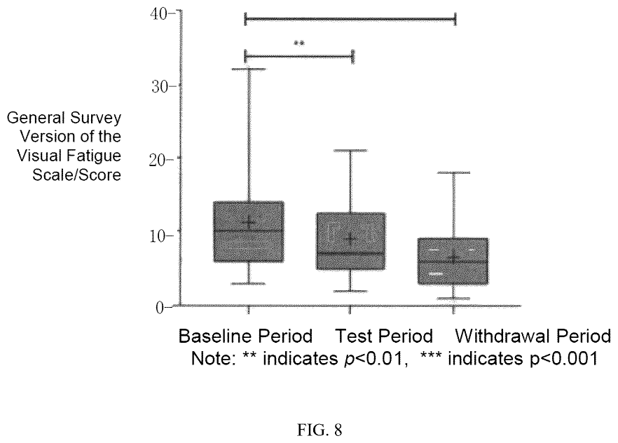

The mean baseline value of the scores of the General Survey Version of the Visual Fatigue Scale for 40 subjects was 11.18, and the mean value at the withdrawal period was 6.60, representing a decrease of 40.94%. There was a significant difference (P=0.0008), as depicted in FIG. 8. After the subjects were administered with the EGT Eye Wash, the mean score of the General Survey Version of Visual Fatigue Scale in the post-treatment period was significantly lower than that in the baseline period (P=0.0008).

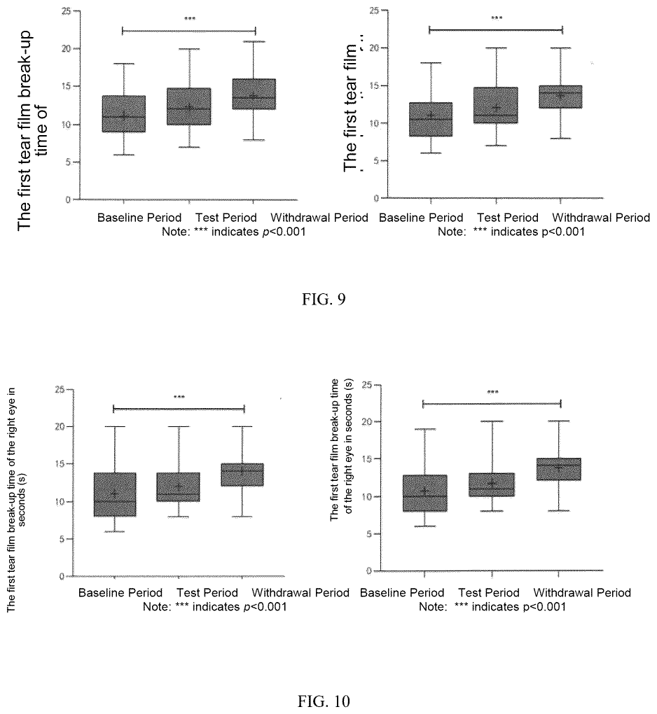

For the 40 subjects, at the withdrawal period, the mean values of the first tear film break-up time of the left eye, the average tear film break-up time of the left eye, the first tear film break-up time of the right eye, and the average tear film break-up time of the right eye increased by 23.60%, 24.56%, 26.98%, and 27.74% respectively compared with those at the baseline period. All of these showed statistically significant differences (P<0.0001, P<0.0001, P<0.0001, P<0.0001), as depicted in FIG. 9 (left eye) and FIG. 10 (right eye). After the subjects were administered with the EGT Eye Wash, the mean values of the first tear film break-up time and the average tear film break-up time of both eyes in the post-treatment period was significantly higher than those in the baseline period (P<0.05).

### Example 3—Formulations

The pharmaceutically acceptable aqueous delivery vehicle was prepared by mixing excipients in water and sterilizing the resulting aqueous solution. EGT was added to the aqueous solution to make eye drops that were 98.0 to 99.5% (w/v) of water. Eye drops of the formulation herein have 0.30%-0.75% (w/v) of EGT, 0.5%-1.0% (w/v) of EGT; 1.0%-2.0% (w/v) of EGT; 0.005% to 0.5% (w/v) of EGT; or 0.005%-0.30% (w/v) of EGT

### Formulation 1

| Component | Amount (% (w/v)) |
|---|---|
| EGT | 0.50-1.00 |
| Aqueous | 98.50-99.0 |
| Delivery | |
| Vehicle | |

### Formulation 2

| Component | Amount (% (w/v)) |
|---|---|
| EGT | 0.30-0.75 |
| Water | 98.50-99.00 |
| Excipients | 0.25-1.20 |

### Formulation 3

| Component | Amount (% (w/v)) |
|---|---|
| EGT | 0.50 |
| Glutathione | 0.10-0.50 |
| Water | 98.50-99.00 |
| Excipients | 0.40-0.50 |

### Formulation 4

| Component | Amount (% (w/v)) |
|---|---|
| EGT | 0.50 |
| Lutein | 0.10-0.50 |
| Water | 98.50-99.00 |
| Excipients | 0.40-0.50 |

### Formulation 5

| Component | Amount (% (w/v)) |
|---|---|
| EGT | 0.50 |
| EDTA | 0.01-0.10 |
| Water | 98.50-99.00 |
| Excipients | 0.40-0.50 |

### Formulation 6

| Component | Amount (% (w/v)) |
|---|---|
| EGT | 0.50 |
| Sodium | 0.05 |
| hyaluronate | |
| Taurine | 0.010 |
| Sodium | 0.50-0.90 |
| chloride | |
| Water or | 98.54-98.84 |
| Aqueous | |
| Delivery | |
| Vehicle | |

### Formulation 7

| Component | Amount (% (w/v)) |
|---|---|
| EGT | 0.50 |
| Sodium | 0.050 |
| hyaluronate | |
| Taurine | 0.03 |
| Sodium | 0.50-0.90 |
| chloride | |
| Water or | 98.52-98.82 |
| Aqueous | |
| Delivery | |
| Vehicle | |

### Formulation 8

| Component | Amount (% (w/v)) |
|---|---|
| EGT | 0.50 |
| Sodium | 0.05 |
| hyaluronate | |
| Taurine | 0.05 |
| Sodium | 0.50-0.90 |
| chloride | |
| Water or | 98.50-98.80 |
| Aqueous | |
| Delivery | |
| Vehicle | |

What is claimed is:

1. A method of treating an oxidative stress-related ocular condition, disorder, or disease in a subject, wherein the method comprises:
administering a formulation to said subject, wherein the formulation comprises L-Ergothioneine (EGT) or a derivative of EGT in a concentration of 0.005% to 2.000% (w/v) in a pharmaceutically acceptable aqueous delivery vehicle,
wherein EGT or the derivative of EGT is either an active ingredient for treating an oxidative stress-related ocular disorder or disease, or an inactive excipient to inhibit degradation of a co-formulated active ingredient to the ocular surface of the subject.

2. The method of claim 1, wherein EGT or the derivative of EGT scavenges reactive oxygen species via a non-enzymatic redox cycle.

3. The method of claim 2, wherein EGT scavenges hydroxyl and peroxynitrite via a non-enzymatic redox cycle, such that:
the hydroxyl is reduced to water and EGT is oxidized to a sulfate; and
the peroxynitrite is reduced to nitrate and EGT is oxidized to a sulfate.

4. The method of claim 1, wherein EGT or the derivative of EGT coordinates to a metal selected from iron, copper, and other transition metals in an ocular surface, thereby inhibiting catalysis of oxidative degradation of labile drugs and decreasing reactive oxygen species (ROS) in corneal epithelial cells.

5. The method of claim 4, further comprising:
forming a complex between EGT and the metal, wherein the metal is in a reduced state; and
reducing an oxidation potential of the metal due to the formation of the complex, thereby suppressing formation of metal-oxygen adducts for deriving reactive oxygen species;
wherein the complex comprises: (i) two units of EGT binding to 1 unit of the metal or (ii) 1 unit of EGT and 1 unit of an initial ligand core binding to 1 unit of the metal.

6. The method of claim 1, wherein the co-formulated active ingredient is selected from the group consisting of: nucleotides, acetylcholine, tetraethylammonium chloride, spermine, L-carnitine, sulpiride, homostachydrine, cytarabine, gemcitabine, gabapentin, oxaliplatin, metformin, cyclosporine, and lifitegrast.

7. The method of claim 1, wherein EGT as the inactive excipient in the formulation is reducing reactive oxygen species (ROS) in corneal epithelial cells.

8. The method of claim 1, wherein:
EGT is 0.5% to 1.0% (w/v) of the formulation, when functioning as an active ingredient;
EGT is 0.005% to 0.5% (w/v) of the formulation, when functioning as an inactive excipient.

9. The method of claim 1, wherein the oxidative stress-related ocular disorder or disease comprises: allergies, irritated eyes, red eye, age-related macular degeneration; cataracts; glaucoma; diabetic retinopathy; dry eye syndrome; and retinitis pigmentosa.

10. The method of claim 1, wherein the co-formulated active ingredient is transported in the ocular tissue by OCTN1, thereby increasing bioavailability of the co-formulated active ingredient in the ocular tissue, wherein OCTN1 is inductively expressed by presence of EGT introduced into the ocular tissue.

11. The method of claim 1, wherein EGT has a chemical formula of C₉H₁₅N₃O₂S, where the carbon-sulfur bond is in thione form (C═S) over thiolate form (C—S⁻), in the pharmaceutically acceptable aqueous delivery vehicle.

12. The method of claim 1, wherein said formulation comprises:
0.5% (w/v) of EGT as the active ingredient;
0.05% (w/v); 0.01% (w/v), and 0% (w/v) of sodium hyaluronate;
trace amounts of taurine;
sodium chloride for adjusting the osmotic pressure of eye drops; and
the pharmaceutically acceptable aqueous delivery vehicle.

13. The method of claim 1, wherein the EGT inhibits degradation of the active ingredient introduced into the ocular tissue, and inductively expresses OCTN1, thereby increasing bioavailability of the active ingredient.

14. A method for treating glaucoma, comprising:
enhancing the trabecular meshwork-Schlemm's canal (TM-SC) pathway to reduce intraocular pressure by administering, to a subject in need thereof, a therapeutically effective amount of a formulation, the formulation comprising:
ergothioneine; and
spermine.

15. The method of claim 14, wherein the formulation further comprises at least one additional ingredient selected from the group consisting of a prostaglandin analog (PGA), a Rho kinase inhibitor, a nitric oxide doner and an adenosine receptor agonist.

16. The method of claim 15, wherein the formulation further comprises at least one chemical chaperone.

17. The method of claim 16, wherein the at least one chemical chaperone includes 4-phenylbutyric acid (4-PBA).

18. The method of claim 16, wherein the at least one chemical chaperone is one of an osmolyte, a hydrophobic compound and a pharmacological chaperone.

19. The method of claim 16, further comprising inhibiting intracellular aggregation of misfolded proteins and restoring trafficking, non-specifically stabilizing native conformations and facilitating escape from endoplasmic reticulum (ER) quality control systems via the chemical chaperone.

20. A method for treating glaucoma, comprising:
inhibiting intracellular aggregation of misfolded proteins and restoring trafficking, non-specifically stabilizing native conformations and facilitating escape from endoplasmic reticulum (ER) quality control systems by administering, to a subject in need thereof, a therapeutically effective amount of a formulation, the formulation comprising:
at least one chemical chaperone selected from the group consisting of an osmolyte, a hydrophobic compound and a pharmacological chaperone.
